(19) United States
(12) Patent Application Publication
(10) Pub. No.: US 2007/0287914 A1
(43) Pub. Date: Dec. 13, 2007

(54) Forward-Looking Intravascular Ultrasound Devices and Methods for Making

(75) Inventor: Adam L. Cohen, Van Nuys, CA (US)

Correspondence Address: MICROFABRICA INC.;ATT: DENNIS R. SMALLEY, 7911 HASKELL AVENUE, VAN NUYS, CA, 91406, US

(73) Assignee: Microfabrica Inc.

(21) Appl. No.: 11/734,271

(22) Filed: Apr. 11, 2007

Related U.S. Application Data
(60) Provisional application No. 60/790,917, filed on Apr. 11, 2006.
(60) Provisional application No. 60/799,455, filed on May 10, 2006.

(51) Int. Cl. A61B 1/00

(52) U.S. Cl. 600/443

(57) ABSTRACT

Embodiments of invention are directed to devices, and methods of forming them, that can be used for imaging interior volumes of the body. Particular embodiments are directed to improved intravascular ultrasound devices. In some embodiments the intravascular ultrasound devices are intended to be ‘forward-looking’; i.e., the image obtained by the device is of structures in front of, or distal to, the device.

### RELATED APPLICATIONS

This application claims benefit of U.S. Provisional Patent Application No. 60/799,455, filed May 10, 2006 and No. 60/790,917, filed Apr. 11, 2006. The ′455 application is incorporated herein by reference as if set forth in full.

### FIELD OF THE INVENTION

The present invention relates to medical devices and in particular to medical devices, typically delivered via a catheter, of the intravascular ultrasound type. In some embodiments these devices may be formed using a multilayer electrochemical fabrication process or the like.

### BACKGROUND

### Background of the Invention

An electrochemical fabrication technique for forming three-dimensional structures from a plurality of adhered layers is being commercially pursued by Microfabrica Inc. (formerly MEMGen® Corporation) of Van Nuys, Calif. under the name EFAB™.

Various electrochemical fabrication techniques were described in U.S. Pat. No. 6,027,630, issued on Feb. 22, 2000 to Adam Cohen. Some embodiments of this electrochemical fabrication technique allows the selective deposition of a material using a mask that includes a patterned conformable material on a support structure that is independent of the substrate onto which plating will occur. When desiring to perform an electrodeposition using the mask, the conformable portion of the mask is brought into contact with a substrate, but not adhered or bonded to the substrate, while in the presence of a plating solution such that the contact of the conformable portion of the mask to the substrate inhibits deposition at selected locations. For convenience, these masks might be generically called conformable contact masks; the masking technique may be generically called a conformable contact mask plating process. More specifically, in the terminology of Microfabrica Inc. such masks have come to be known as INSTANT MASKS™ and the process known as INSTANT MASKNG™ or INSTANT MASK™ plating. Selective depositions using conformable contact mask plating may be used to form single selective deposits of material or may be used in a process to form multi-layer structures. The teachings of the '630 patent are hereby incorporated herein by reference as if set forth in full herein. Since the filing of the patent application that led to the above noted patent, various papers about conformable contact mask plating (i.e. INSTANT MASKING) and electrochemical fabrication have been published:

(1) A. Cohen, G. Zhang, F. Tseng, F. Mansfeld, U. Frodis and P. Will, “EFAB: Batch production of functional, fully-dense metal parts with micro-scale features”, Proc. 9th Solid Freeform Fabrication, The University of Texas at Austin, p 161, Aug. 1998.

(2) A. Cohen, G. Zhang, F. Tseng, F. Mansfeld, U. Frodis and P. Will, “EFAB: Rapid, Low-Cost Desktop Micromachining of High Aspect Ratio True 3-D MEMS”, Proc. 12th IEEE Micro Electro Mechanical Systems Workshop, IEEE, p 244, January 1999.

(3) A. Cohen, “3-D Micromachining by Electrochemical Fabrication”, Micromachine Devices, March 1999.

(4) G. Zhang, A. Cohen, U. Frodis, F. Tseng, F. Mansfeld, and P. Will, “EFAB: Rapid Desktop Manufacturing of True 3-D Microstructures”, Proc. 2nd International Conference on Integrated MicroNanotechnology for Space Applications, The Aerospace Co., Apr. 1999.

(5) F. Tseng, U. Frodis, G. Zhang, A. Cohen, F. Mansfeld, and P. Will, “EFAB: High Aspect Ratio, Arbitrary 3-D Metal Microstructures using a Low-Cost Automated Batch Process”, 3rd International Workshop on High Aspect Ratio MicroStructure Technology (HARMST'99), June 1999.

(6) A. Cohen, U. Frodis, F. Tseng, G. Zhang, F. Mansfeld, and P. Will, “EFAB: Low-Cost, Automated Electrochemical Batch Fabrication of Arbitrary 3-D Microstructures”, Micromachining and Microfabrication Process Technology, SPIE 1999 Symposium on Micromachining and Microfabrication, September 1999.

(7) F. Tseng, G. Zhang, U. Frodis, A. Cohen, F. Mansfeld, and P. Will, “EFAB: High Aspect Ratio, Arbitrary 3-D Metal Microstructures using a Low-Cost Automated Batch Process”, MEMS Symposium, ASME 1999 International Mechanical Engineering Congress and Exposition, November, 1999.

(8) A. Cohen, “Electrochemical Fabrication (EFAB™)”, Chapter 19 of The MEMS Handbook, edited by Mohamed Gad-El-Hak, CRC Press, 2002.

(9) Microfabrication—Rapid Prototyping's Killer Application”, pages 1-5 of the Rapid Prototyping Report, CAD/CAM Publishing, Inc., June 1999.

The disclosures of these nine publications are hereby incorporated herein by reference as if set forth in full herein.

An electrochemical deposition for forming multilayer structures may be carried out in a number of different ways as set forth in the above patent and publications. In one form, this process involves the execution of three separate operations during the formation of each layer of the structure that is to be formed:
1. Selectively depositing at least one material by electrodeposition upon one or more desired regions of a substrate. Typically this material is either a structural material or a sacrificial material.
2. Then, blanket depositing at least one additional material by electrodeposition so that the additional deposit covers both the regions that were previously selectively deposited onto, and the regions of the substrate that did not receive any previously applied selective depositions. Typically this material is the other of a structural material or a sacrificial material.
3. Finally, planarizing the materials deposited during the first and second operations to produce a smoothed surface of a first layer of desired thickness having at least one region containing the at least one material and at least one region containing at least the one additional material.

After formation of the first layer, one or more additional layers may be formed adjacent to an immediately preceding layer and adhered to the smoothed surface of that preceding layer. These additional layers are formed by repeating the first through third operations one or more times wherein the formation of each subsequent layer treats the previously formed layers and the initial substrate as a new and thickening substrate.

Once the formation of all layers has been completed, at least a portion of at least one of the materials deposited is generally removed by an etching process to expose or release the three-dimensional structure that was intended to be formed. The removed material is a sacrificial material while the material that forms part of the desired structure is a structural material.

The preferred method of performing the selective electrodeposition involved in the first operation is by conformable contact mask plating. In this type of plating, one or more conformable contact (CC) masks are first formed. The CC masks include a support structure onto which a patterned conformable dielectric material is adhered or formed. The conformable material for each mask is shaped in accordance with a particular cross-section of material to be plated (the pattern of conformable material is complementary to the pattern of material to be deposited). At least one CC mask is used for each unique cross-sectional pattern that is to be plated.

The support for a CC mask is typically a plate-like structure formed of a metal that is to be selectively electroplated and from which material to be plated will be dissolved. In this typical approach, the support will act as an anode in an electroplating process. In an alternative approach, the support may instead be a porous or otherwise perforated material through which deposition material will pass during an electroplating operation on its way from a distal anode to a deposition surface. In either approach, it is possible for multiple CC masks to share a common support, i.e. the patterns of conformable dielectric material for plating multiple layers of material may be located in different areas of a single support structure. When a single support structure contains multiple plating patterns, the entire structure is referred to as the CC mask while the individual plating masks may be referred to as “submasks”. In the present application such a distinction will be made only when relevant to a specific point being made.

In preparation for performing the selective deposition of the first operation, the conformable portion of the CC mask is placed in registration with and pressed against a selected portion of (1) the substrate, (2) a previously formed layer, or (3) a previously deposited portion of a layer on which deposition is to occur. The pressing together of the CC mask and relevant substrate occur in such a way that all openings, in the conformable portions of the CC mask contain plating solution. The conformable material of the CC mask that contacts the substrate acts as a barrier to electrodeposition while the openings in the CC mask that are filled with electroplating solution act as pathways for transferring material from an anode (e.g. the CC mask support) to the non-contacted portions of the substrate (which act as a cathode during the plating operation) when an appropriate potential and/or current are supplied.

An example of a CC mask and CC mask plating are shown in FIGS. 1A-1C. FIG. 1A shows a side view of a CC mask 8 consisting of a conformable or deformable (e.g. elastomeric) insulator 10 patterned on an anode 12. The anode has two functions. One is as a supporting material for the patterned insulator 10 to maintain its integrity and alignment since the pattern may be topologically complex (e.g., involving isolated “islands” of insulator material). The other function is as an anode for the electroplating operation. FIG. 1A also depicts a substrate 6, separated from mask 8, onto which material will be deposited during the process of forming a layer. CC mask plating selectively deposits material 22 onto substrate 6 by simply pressing the insulator against the substrate then electrodepositing material through apertures 26a and 26b in the insulator as shown in FIG. 1B. After deposition, the CC mask is separated, preferably non-destructively, from the substrate 6 as shown in FIG. 1C.

The CC mask plating process is distinct from a “through-mask” plating process in that in a through-mask plating process the separation of the masking material from the substrate would occur destructively. Furthermore in a through mask plating process, opening in the masking material are typically formed while the masking material is in contact with and adhered to the substrate. As with through-mask plating, CC mask plating deposits material selectively and simultaneously over the entire layer. The plated region may consist of one or more isolated plating regions where these isolated plating regions may belong to a single structure that is being formed or may belong to multiple structures that are being formed simultaneously. In CC mask plating as individual masks are not intentionally destroyed in the removal process, they may be usable in multiple plating operations.

Another example of a CC mask and CC mask plating is shown in FIGS. 1D-1G. FIG. 1D shows an anode 12′ separated from a mask 8′ that includes a patterned conformable material 10′ and a support structure 20. FIG. 1D also depicts substrate 6 separated from the mask 8′. FIG. 1E illustrates the mask 8′ being brought into contact with the substrate 6. FIG. 1F illustrates the deposit 22′ that results from conducting a current from the anode 12′ to the substrate 6. FIG. 1G illustrates the deposit 22′ on substrate 6 after separation from mask 8′. In this example, an appropriate electrolyte is located between the substrate 6 and the anode 12′ and a current of ions coming from one or both of the solution and the anode are conducted through the opening in the mask to the substrate where material is deposited. This type of mask may be referred to as an anodeless INSTANT MASK™ (AIM) or as an anodeless conformable contact (ACC) mask.

Unlike through-mask plating, CC mask plating allows CC masks to be formed completely separate from the substrate on which plating is to occur (e.g. separate from a three-dimensional (3D) structure that is being formed). CC masks may be formed in a variety of ways, for example, using a photolithographic process. All masks can be generated simultaneously, e.g. prior to structure fabrication rather than during it. This separation makes possible a simple, low-cost, automated, self-contained, and internally-clean “desktop factory” that can be installed almost anywhere to fabricate 3D structures, leaving any required clean room processes, such as photolithography to be performed by service bureaus or the like.

An example of the electrochemical fabrication process discussed above is illustrated in FIGS. 2A-2F. These figures show that the process involves deposition of a first material 2 which is a sacrificial material and a second material 4 which is a structural material. The CC mask 8, in this example, includes a patterned conformable material (e.g. an elastomeric dielectric material) 10 and a support 12 which is made from deposition material 2. The conformal portion of the CC mask is pressed against substrate 6 with a plating solution 14 located within the openings 16 in the conformable material 10. An electric current, from power supply 18, is then passed through the plating solution 14 via (a) support 12 which doubles as an anode and (b) substrate 6 which doubles as a cathode. FIG. 2A illustrates that the passing of current causes material 2 within the plating solution and material 2 from the anode 12 to be selectively transferred to and plated on the substrate 6. After electroplating the first deposition material 2 onto the substrate 6 using CC mask 8, the CC mask 8 is removed as shown in FIG. 2B. FIG. 2C depicts the second deposition material 4 as having been blanket-deposited (i.e. non-selectively deposited) over the previously deposited first deposition material 2 as well as over the other portions of the substrate 6. The blanket deposition occurs by electroplating from an anode (not shown), composed of the second material, through an appropriate plating solution (not shown), and to the cathode/substrate 6. The entire two-material layer is then planarized to achieve precise thickness and flatness as shown in FIG. 2D. After repetition of this process for all layers, the multi-layer structure 20 formed of the second material 4 (i.e. structural material) is embedded in first material 2 (i.e. sacrificial material) as shown in FIG. 2E. The embedded structure is etched to yield the desired device, i.e. structure 20, as shown in FIG. 2F.

Various components of an exemplary manual electrochemical fabrication system 32 are shown in FIGS. 3A-3C. The system 32 consists of several subsystems 34, 36, 38, and 40. The substrate holding subsystem 34 is depicted in the upper portions of each of FIGS. 3A-3C and includes several components: (1) a carrier 48, (2) a metal substrate 6 onto which the layers are deposited, and (3) a linear slide 42 capable of moving the substrate 6 up and down relative to the carrier 48 in response to drive force from actuator 44. Subsystem 34 also includes an indicator 46 for measuring differences in vertical position of the substrate which may be used in setting or determining layer thicknesses and/or deposition thicknesses. The subsystem 34 further includes feet 68 for carrier 48 which can be precisely mounted on subsystem 36.

The CC mask subsystem 36 shown in the lower portion of FIG. 3A includes several components: (1) a CC mask 8 that is actually made up of a number of CC masks (i.e. submasks) that share a common support/anode 12, (2) precision X-stage 54, (3) precision Y-stage 56, (4) frame 72 on which the feet 68 of subsystem 34 can mount, and (5) a tank 58 for containing the electrolyte 16. Subsystems 34 and 36 also include appropriate electrical connections (not shown) for connecting to an appropriate power source (not shown) for driving the CC masking process.

The blanket deposition subsystem 38 is shown in the lower portion of FIG. 3B and includes several components: (1) an anode 62, (2) an electrolyte tank 64 for holding plating solution 66, and (3) frame 74 on which feet 68 of subsystem 34 may sit. Subsystem 38 also includes appropriate electrical connections (not shown) for connecting the anode to an appropriate power supply (not shown) for driving the blanket deposition process.

The planarization subsystem 40 is shown in the lower portion of FIG. 3C and includes a lapping plate 52 and associated motion and control systems (not shown) for planarizing the depositions.

In addition to teaching the use of CC masks for electrodeposition purposes, the '630 patent also teaches that the CC masks may be placed against a substrate with the polarity of the voltage reversed and material may thereby be selectively removed from the substrate. It indicates that such removal processes can be used to selectively etch, engrave, and polish a substrate, e.g., a plaque.

The '630 patent further indicates that the electroplating methods and articles disclosed therein allow fabrication of devices from thin layers of materials such as, e.g., metals, polymers, ceramics, and semiconductor materials. It further indicates that although the electroplating embodiments described therein have been described with respect to the use of two metals, a variety of materials, e.g., polymers, ceramics and semiconductor materials, and any number of metals can be deposited either by the electroplating methods therein, or in separate processes that occur throughout the electroplating method. It indicates that a thin plating base can be deposited, e.g., by sputtering, over a deposit that is insufficiently conductive (e.g., an insulating layer) so as to enable subsequent electroplating. It also indicates that multiple support materials (i.e. sacrificial materials) can be included in the electroplated element allowing selective removal of the support materials.

The '630 patent additionally teaches that the electroplating methods disclosed therein can be used to manufacture elements having complex microstructure and close tolerances between parts. An example is given with the aid of FIGS. 14A-14E of that patent. In the example, elements having parts that fit with close tolerances, e.g., having gaps between about 1-5 um, including electroplating the parts of the device in an unassembled, preferably pre-aligned, state and once fabricated. In such embodiments, the individual parts can be moved into operational relation with each other or they can simply fall together. Once together the separate parts may be retained by clips or the like.

Another method for forming microstructures from electroplated metals (i.e. using electrochemical fabrication techniques) is taught in U.S. Pat. No. 5,190,637 to Henry Guckel, entitled “Formation of Microstructures by Multiple Level Deep X-ray Lithography with Sacrificial Metal layers”. This patent teaches the formation of metal structure utilizing through mask exposures. A first layer of a primary metal is electroplated onto an exposed plating base to fill a void in a photoresist (the photoresist forming a through mask having a desired pattern of openings), the photoresist is then removed and a secondary metal is electroplated over the first layer and over the plating base. The exposed surface of the secondary metal is then machined down to a height which exposes the first metal to produce a flat uniform surface extending across both the primary and secondary metals. Formation of a second layer may then begin by applying a photoresist over the first layer and patterning it (i.e. to form a second through mask) and then repeating the process that was used to produce the first layer to produce a second layer of desired configuration. The process is repeated until the entire structure is formed and the secondary metal is removed by etching. The photoresist is formed over the plating base or previous layer by casting and patterning of the photoresist (i.e. voids formed in the photoresist) are formed by exposure of the photoresist through a patterned mask via X-rays or UV radiation and development of the exposed or unexposed areas.

The '637 patent teaches the locating of a plating base onto a substrate in preparation for electroplating materials onto the substrate. The plating base is indicated as typically involving the use of a sputtered film of an adhesive metal, such as chromium or titanium, and then a sputtered film of the metal that is to be plated. It is also taught that the plating base may be applied over an initial layer of sacrificial material (i.e. a layer or coating of a single material) on the substrate so that the structure and substrate may be detached if desired. In such cases after formation of the structure the sacrificial material forming part of each layer of the structure may be removed along the initial sacrificial layer to free the structure. Substrate materials mentioned in the '637 patent include silicon, glass, metals, and silicon with protected semiconductor devices. A specific example of a plating base includes about 150 angstroms of titanium and about 300 angstroms of nickel, both of which are sputtered at a temperature of 160° C. In another example it is indicated that the plating base may consist of 150 angstroms of titanium and 150 angstroms of nickel where both are applied by sputtering.

Electrochemical Fabrication provides the ability to form prototypes and commercial quantities of miniature objects, parts, structures, devices, and the like at reasonable costs and in reasonable times. In fact, Electrochemical Fabrication is an enabler for the formation of many structures that were hitherto impossible to produce. Electrochemical Fabrication opens the spectrum for new designs and products in many industrial fields. Even though Electrochemical Fabrication offers this new capability and it is understood that Electrochemical Fabrication techniques can be combined with designs and structures known within various fields to produce new structures, certain uses for Electrochemical Fabrication provide designs, structures, capabilities and/or features not known or obvious in view of the state of the art.

A need exists in various fields for miniature devices having improved characteristics, reduced fabrication times, reduced fabrication costs, simplified fabrication processes, greater versatility in device design, improved selection of materials, improved material properties, more cost effective and less risky production of such devices, and/or more independence between geometric configuration and the selected fabrication process.

### SUMMARY OF THE INVENTION

It is an object of some embodiments of the invention to provide improved devices for imaging interior volumes of the body.

It is an object of some embodiments of the invention to provide improved intravascular ultrasound devices.

It is an object of some embodiments of the invention to provide improved methods for forming intravascular ultrasound devices.

Other objects and advantages of various embodiments of the invention will be apparent to those of skill in the art upon review of the teachings herein. The various embodiments of the invention, set forth explicitly herein or otherwise ascertained from the teachings herein, may address one or more of the above objects alone or in combination, or alternatively may address some other object ascertained from the teachings herein. It is not necessarily intended that all objects be addressed by any single aspect of the invention even though that may be the case with regard to some aspects.

A first aspect of the invention provides an intravascular imaging device, including: a catheter having a proximal and distal end; a scanning mechanism having an proximal end and a distal end, wherein the proximal is functionally connected to the distal end of the catheter; a drive cable located within the catheter and functionally connected to scanning mechanism; wherein the scanning mechanism including: a signal transducer, a mechanical mechanism for scanning the transducer that caused the scanning mechanism to scan a pattern which is different from a motion carried by the drive cable.

Other aspects of the invention will be understood by those of skill in the art upon review of the teachings herein. These other aspects of the invention may provide various combinations of the aspects presented above as well as provide other configurations, structures, functional relationships, and processes that have not been specifically set forth above.

### BRIEF DESCRIPTION OF THE DRAWINGS

FIGS. 1A-1C schematically depict side views of various stages of a CC mask plating process, while FIGS. 1D-G schematically depict a side views of various stages of a CC mask plating process using a different type of CC mask.

FIGS. 2A-2F schematically depict side views of various stages of an electrochemical fabrication process as applied to the formation of a particular structure where a sacrificial material is selectively deposited while a structural material is blanket deposited.

FIGS. 3A-3C schematically depict side views of various example subassemblies that may be used in manually implementing the electrochemical fabrication method depicted in FIGS. 2A-2F.

FIGS. 4A-4F schematically depict the formation of a first layer of a structure using adhered mask plating where the blanket deposition of a second material overlays both the openings between deposition locations of a first material and the first material itself

FIG. 4G depicts the completion of formation of the first layer resulting from planarizing the deposited materials to a desired level.

FIGS. 4H and 4I respectively depict the state of the process after formation of the multiple layers of the structure and after release of the structure from the sacrificial material.

FIG. 5 provides a partially cut perspective view of the device 102 of the first embodiment.

FIGS. 6-7 provide partially cut views focusing on the distal end of the device of FIG. 5.

FIGS. 8-18 provide views of the mechanism of the device from various perspectives and with various components removed for clarity.

FIGS. 19-21 provide various perspective, sectional views along various planes, of the mechanism with the drive cable, transducer, and bond wire also shown.

FIGS. 22A-22E depict various states in an example process according to which dielectric structural material may be incorporated into a device produced using an electrochemical fabrication technology, and in particular, they illustrate how a dielectric structural material may form a portion of the carriage of FIGS. 5-21 as described above.

FIG. 23A-33F illustrate various states of an electrochemical fabrication process or forming the fulcrum portions of the devices of FIGS. 5-21.

FIGS. 24 and 25 provide top views of a carriage element and a carriage element holding multiple transducers according to an alternative embodiment of the invention.

### DETAILED DESCRIPTION OF PREFERRED EMBODIMENTS

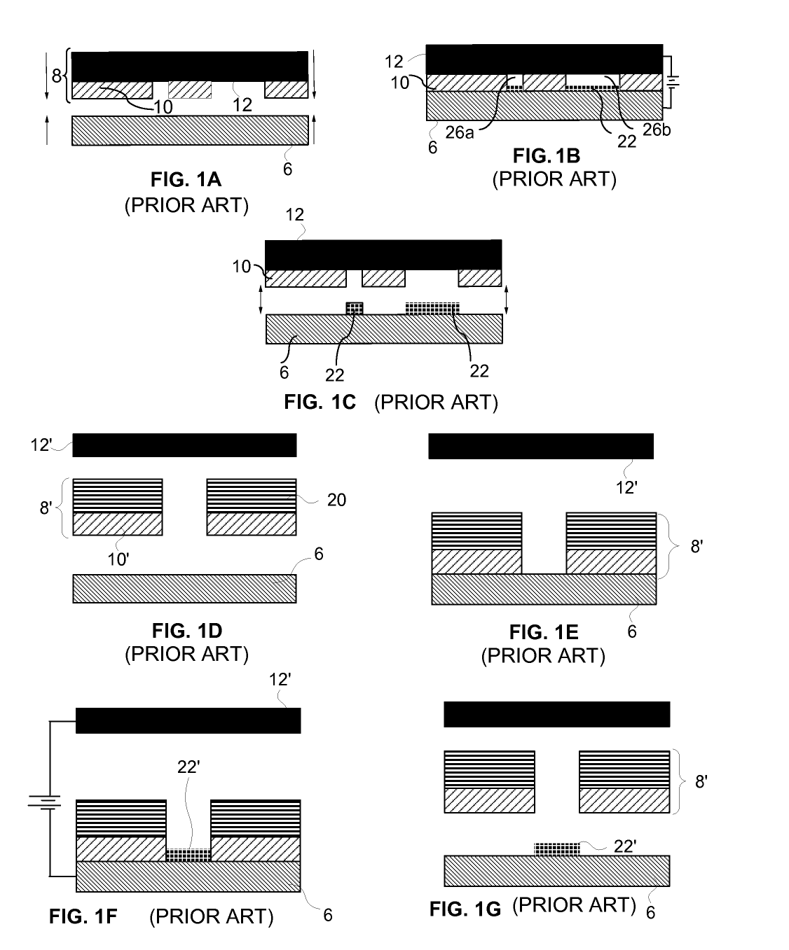

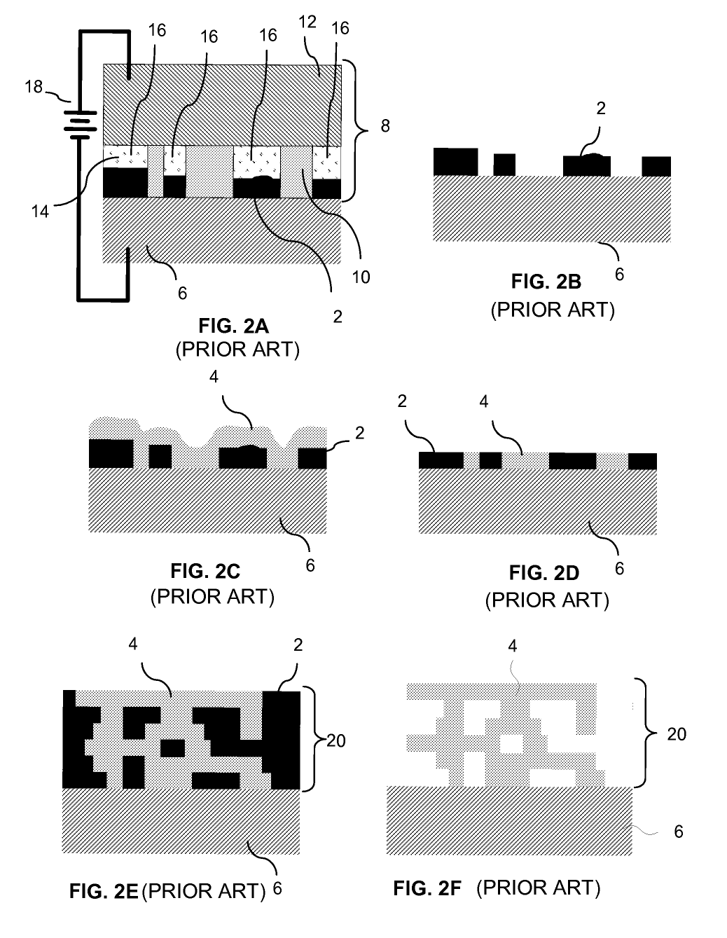

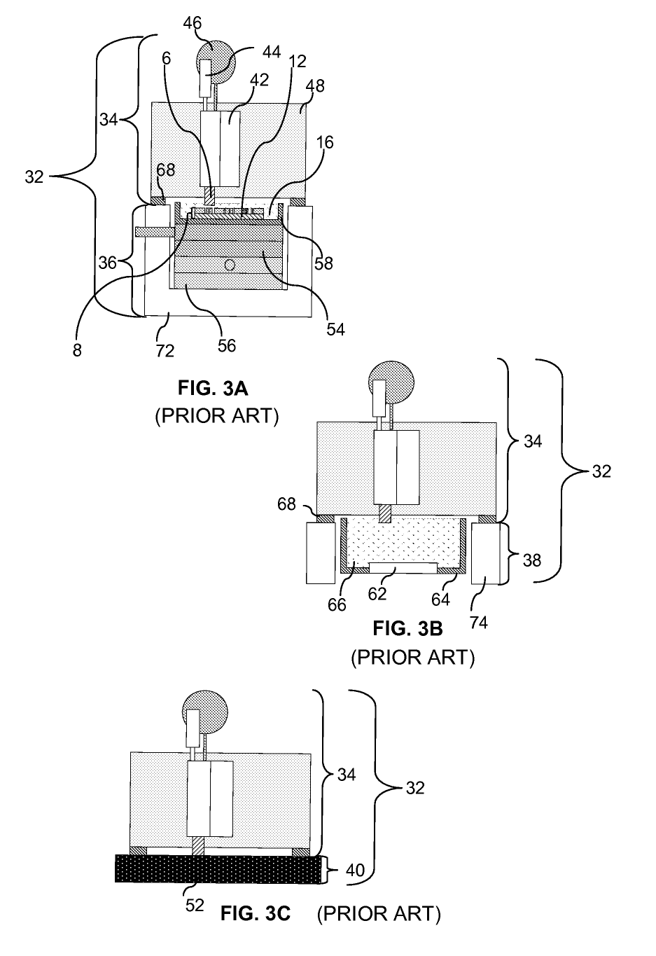

FIGS. 1A-1G, 2A-2F, and 3A-3C illustrate various features of one form of electrochemical fabrication. Other electrochemical fabrication techniques are set forth in the '630 patent referenced above, in the various previously incorporated publications, in various other patents and patent applications incorporated herein by reference. Still others may be derived from combinations of various approaches described in these publications, patents, and applications, or are otherwise known or ascertainable by those of skill in the art from the teachings set forth herein. All of these techniques may be combined with those of the various embodiments of various aspects of the invention to yield enhanced embodiments. Still other embodiments may be derived from combinations of the various embodiments explicitly set forth herein.

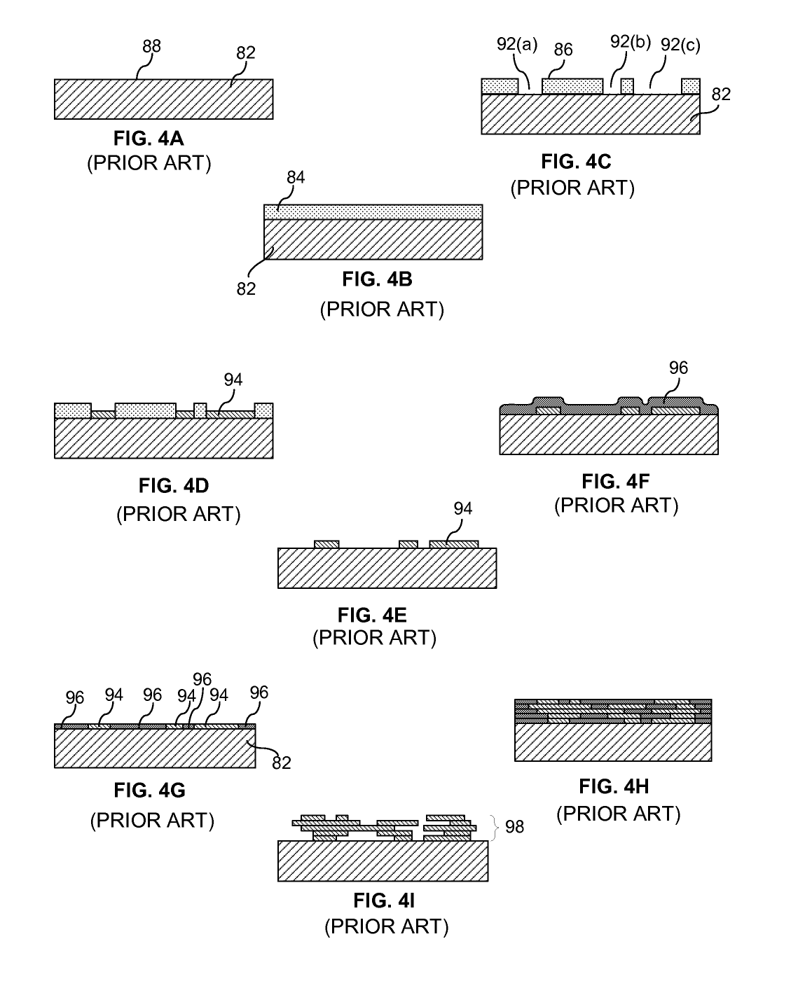

FIGS. 4A-4I illustrate various stages in the formation of a single layer of a multi-layer fabrication process where a second metal is deposited on a first metal as well as in openings in the first metal so that the first and second metal form part of the layer. In FIG. 4A a side view of a substrate 82 is shown, onto which patternable photoresist 84 is cast as shown in FIG. 4B. In FIG. 4C, a pattern of resist is shown that results from the curing, exposing, and developing of the resist. The patterning of the photoresist 84 results in openings or apertures 92(a)-92(c) extending from a surface 86 of the photoresist through the thickness of the photoresist to surface 88 of the substrate 82. In FIG. 4D a metal 94 (e.g. nickel) is shown as having been electroplated into the openings 92(a)-92(c). In FIG. 4E the photoresist has been removed (i.e. chemically stripped) from the substrate to expose regions of the substrate 82 which are not covered with the first metal 94. In FIG. 4F a second metal 96 (e.g. silver) is shown as having been blanket electroplated over the entire exposed portions of the substrate 82 (which is conductive) and over the first metal 94 (which is also conductive). FIG. 4G depicts the completed first layer of the structure which has resulted from the planarization of the first and second metals down to a height that exposes the first metal and sets a thickness for the first layer. In FIG. 4H the result of repeating the process steps shown in FIGS. 4B-4 G several times to form a multi-layer structure are shown where each layer consists of two materials. For most applications, one of these materials is removed as shown in FIG. 4I to yield a desired 3-D structure 98 (e.g. component or device).

Various embodiments of various aspects of the invention are directed to formation of three-dimensional structures from materials some of which may be electrodeposited or electroless deposited. Some of these structures may be formed form a single build level formed from one or more deposited materials while others are formed from a plurality of build layers each including at least two materials (e.g. two or more layers, more preferably five or more layers, and most preferably ten or more layers). In some embodiments, layer thicknesses may be as small as one micron or as large as fifty microns. In other embodiments, thinner layers may be used while in other embodiments, thicker layers may be used. In some embodiments structures having features positioned with micron level precision and minimum features size on the order of tens of microns are to be formed. In other embodiments structures with less precise feature placement and/or larger minimum features may be formed. In still other embodiments, higher precision and smaller minimum feature sizes may be desirable.

The various embodiments, alternatives, and techniques disclosed herein may form multi-layer structures using a single patterning technique on all layers or using different patterning techniques on different layers. For example, Various embodiments of the invention may perform selective patterning operations using conformable contact masks and masking operations (i.e. operations that use masks which are contacted to but not adhered to a substrate), proximity masks and masking operations (i.e. operations that use masks that at least partially selectively shield a substrate by their proximity to the substrate even if contact is not made), non-conformable masks and masking operations (i.e. masks and operations based on masks whose contact surfaces are not significantly conformable), and/or adhered masks and masking operations (masks and operations that use masks that are adhered to a substrate onto which selective deposition or etching is to occur as opposed to only being contacted to it). Conformable contact masks, proximity masks, and non-conformable contact masks share the property that they are preformed and brought to, or in proximity to, a surface which is to be treated (i.e. the exposed portions of the surface are to be treated). These masks can generally be removed without damaging the mask or the surface that received treatment to which they were contacted, or located in proximity to. Adhered masks are generally formed on the surface to be treated (i.e. the portion of that surface that is to be masked) and bonded to that surface such that they cannot be separated from that surface without being completely destroyed damaged beyond any point of reuse. Adhered masks may be formed in a number of ways including (1) by application of a photoresist, selective exposure of the photoresist, and then development of the photoresist, (2) selective transfer of pre-patterned masking material, and/or (3) direct formation of masks from computer controlled depositions of material.

Patterning operations may be used in selectively depositing material and/or may be used in the selective etching of material. Selectively etched regions may be selectively filled in or filled in via blanket deposition, or the like, with a different desired material. In some embodiments, the layer-by-layer build up may involve the simultaneous formation of portions of multiple layers. In some embodiments, depositions made in association with some layer levels may result in depositions to regions associated with other layer levels (i.e. regions that lie within the top and bottom boundary levels that define a different layer's geometric configuration). Such use of selective etching and interlaced material deposition in association with multiple layers is described in U.S. patent application Ser. No. 10/434,519, by Smalley, and entitled “Methods of and Apparatus for Electrochemically Fabricating Structures Via Interlaced Layers or Via Selective Etching and Filling of Voids layer elements” which is hereby incorporated herein by reference as if set forth in full.

Temporary substrates on which structures may be formed may be of the sacrificial-type (i.e. destroyed or damaged during separation of deposited materials to the extent they can not be reused), non-sacrificial-type (i.e. not destroyed or excessively damaged, i.e. not damaged to the extent they may not be reused, e.g. with a sacrificial or release layer located between the substrate and the initial layers of a structure that is formed). Non-sacrificial substrates may be considered reusable, with little or no rework (e.g. replanarizing one or more selected surfaces or applying a release layer, and the like) though they may or may not be reused for a variety of reasons.

Definitions

This section of the specification is intended to set forth definitions for a number of specific terms that may be useful in describing the subject matter of the various embodiments of the invention. It is believed that the meanings of most if not all of these terms is clear from their general use in the specification but they are set forth hereinafter to remove any ambiguity that may exist. It is intended that these definitions be used in understanding the scope and limits of any claims that use these specific terms. As far as interpretation of the claims of this patent disclosure are concerned, it is intended that these definitions take presence over any contradictory definitions or allusions found in any materials which are incorporated herein by reference.

“Build” as used herein refers, as a verb, to the process of building a desired structure or plurality of structures from a plurality of applied or deposited materials which are stacked and adhered upon application or deposition or, as a noun, to the physical structure or structures formed from such a process. Depending on the context in which the term is used, such physical structures may include a desired structure embedded within a sacrificial material or may include only desired physical structures which may be separated from one another or may require dicing and/or slicing to cause separation.

“Build axis” or “build orientation” is the axis or orientation that is substantially perpendicular to substantially planar levels of deposited or applied materials that are used in building up a structure. The planar levels of deposited or applied materials may be or may not be completely planar but are substantially so in that the overall extent of their cross-sectional dimensions are significantly greater than the height of any individual deposit or application of material (e.g. 100, 500, 1000, 5000, or more times greater). The planar nature of the deposited or applied materials may come about from use of a process that leads to planar deposits or it may result from a planarization process (e.g. a process that includes mechanical abrasion, e.g. lapping, fly cutting, grinding, or the like) that is used to remove material regions of excess height. Unless explicitly noted otherwise, “vertical” as used herein refers to the build axis or nominal build axis (if the layers are not stacking with perfect registration) while “horizontal” refers to a direction within the plane of the layers (i.e. the plane that is substantially perpendicular to the build axis).

“Build layer” or “layer of structure” as used herein does not refer to a deposit of a specific material but instead refers to a region of a build located between a lower boundary level and an upper boundary level which generally defines a single cross-section of a structure being formed or structures which are being formed in parallel. Depending on the details of the actual process used to form the structure, build layers are generally formed on and adhered to previously formed build layers. In some processes the boundaries between build layers are defined by planarization operations which result in successive build layers being formed on substantially planar upper surfaces of previously formed build layers. In some embodiments, the substantially planar upper surface of the preceding build layer may be textured to improve adhesion between the layers. In other build processes, openings may exist in or be formed in the upper surface of a previous but only partially formed build layers such that the openings in the previous build layers are filled with materials deposited in association with current build layers which will cause interlacing of build layers and material deposits. Such interlacing is described in U.S. patent application Ser. No. 10/434,519. This referenced application is incorporated herein by reference as if set forth in full. In most embodiments, a build layer includes at least one primary structural material and at least one primary sacrificial material. However, in some embodiments, two or more primary structural materials may used without a primary sacrificial material (e.g. when one primary structural material is a dielectric and the other is a conductive material). In some embodiments, build layers are distinguishable from each other by the source of the data that is used to yield patterns of the deposits, applications, and/or etchings of material that form the respective build layers. For example, data descriptive of a structure to be formed which is derived from data extracted from different vertical levels of a data representation of the structure define different build layers of the structure. The vertical separation of successive pairs of such descriptive data may define the thickness of build layers associated with the data. As used herein, at times, “build layer” may be loosely referred simply as “layer”. In many embodiments, deposition thickness of primary structural or sacrificial materials (i.e. the thickness of any particular material after it is deposited) is generally greater than the layer thickness and a net deposit thickness is set via one or more planarization processes which may include, for example, mechanical abrasion (e.g. lapping, fly cutting, polishing, and the like) and/or chemical etching (e.g. using selective or non-selective etchants). The lower boundary and upper boundary for a build layer may be set and defined in different ways. From a design point of view they may be set based on a desired vertical resolution of the structure (which may vary with height). From a data manipulation point of view, the vertical layer boundaries may be defined as the vertical levels at which data descriptive of the structure is processed or the layer thickness may be defined as the height separating successive levels of cross-sectional data that dictate how the structure will be formed. From a fabrication point of view, depending on the exact fabrication process used, the upper and lower layer boundaries may be defined in a variety of different ways. For example by planarization levels or effective planarization levels (e.g. lapping levels, fly cutting levels, chemical mechanical polishing levels, mechanical polishing levels, vertical positions of structural and/or sacrificial materials after relatively uniform etch back following a mechanical or chemical mechanical planarization process). For example, by levels at which process steps or operations are repeated. At levels at which, at least theoretically, lateral extends of structural material can be changed to define new cross-sectional features of a structure.

“Layer thickness” is the height along the build axis between a lower boundary of a build layer and an upper boundary of that build layer.

“Planarization” is a process that tends to remove materials, above a desired plane, in a substantially non-selective manner such that all deposited materials are brought to a substantially common height or desired level (e.g. within 20%, 10%, 5%, or even 1% of a desired layer boundary level). For example, lapping removes material in a substantially non-selective manner though some amount of recession one material or another may occur (e.g. copper may recess relative to nickel). Planarization may occur primarily via mechanical means, e.g. lapping, grinding, fly cutting, milling, sanding, abrasive polishing, frictionally induced melting, other machining operations, or the like (i.e. mechanical planarization). Mechanical planarization maybe followed or proceeded by thermally induced planarization (.e.g. melting) or chemically induced planarization (e.g. etching). Planarization may occur primarily via a chemical and/or electrical means (e.g. chemical etching, electrochemical etching, or the like). Planarization may occur via a simultaneous combination of mechanical and chemical etching (e.g. chemical mechanical polishing (CMP)).

“Structural material” as used herein refers to a material that remains part of the structure when put into use.

“Supplemental structural material” as used herein refers to a material that forms part of the structure when the structure is put to use but is not added as part of the build layers but instead is added to a plurality of layers simultaneously (e.g. via one or more coating operations that applies the material, selectively or in a blanket fashion, to a one or more surfaces of a desired build structure that has been released from a sacrificial material.

“Primary structural material” as used herein is a structural material that forms part of a given build layer and which is typically deposited or applied during the formation of that build layer and which makes up more than 20% of the structural material volume of the given build layer. In some embodiments, the primary structural material may be the same on each of a plurality of build layers or it may be different on different build layers. In some embodiments, a given primary structural material may be formed from two or more materials by the alloying or diffusion of two or more materials to form a single material.

“Secondary structural material” as used herein is a structural material that forms part of a given build layer and is typically deposited or applied during the formation of the given build layer but is not a primary structural material as it individually accounts for only a small volume of the structural material associated with the given layer. A secondary structural material will account for less than 20% of the volume of the structural material associated with the given layer. In some preferred embodiments, each secondary structural material may account for less than 10%, 5%, or even 2% of the volume of the structural material associated with the given layer. Examples of secondary structural materials may include seed layer materials, adhesion layer materials, barrier layer materials (e.g. diffusion barrier material), and the like. These secondary structural materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931. In other embodiments, such coatings may be applied in a non-planar manner, for example, in openings in and over a patterned masking material that has been applied to previously planarized layers of material as taught in U.S. patent application Ser. No. 10/841,383. These referenced applications are incorporated herein by reference as if set forth in full herein.

“Functional structural material” as used herein is a structural material that would have been removed as a sacrificial material but for its actual or effective encapsulation by other structural materials. Effective encapsulation refers, for example, to the inability of an etchant to attack the functional structural material due to inaccessibility that results from a very small area of exposure and/or due to an elongated or tortuous exposure path. For example, large (10,000 μm²) but thin (e.g. less than 0.5 microns) regions of sacrificial copper sandwiched between deposits of nickel may define regions of functional structural material depending on ability of a release etchant to remove the sandwiched copper.

“Sacrificial material” is material that forms part of a build layer but is not a structural material. Sacrificial material on a given build layer is separated from structural material on that build layer after formation of that build layer is completed and more generally is removed from a plurality of layers after completion of the formation of the plurality of layers during a “release” process that removes the bulk of the sacrificial material or materials. In general sacrificial material is located on a build layer during the formation of one, two, or more subsequent build layers and is thereafter removed in a manner that does not lead to a planarized surface. Materials that are applied primarily for masking purposes, i.e. to allow subsequent selective deposition or etching of a material, e.g. photoresist that is used in forming a build layer but does not form part of the build layer) or that exist as part of a build for less than one or two complete build layer formation cycles are not considered sacrificial materials as the term is used herein but instead shall be referred as masking materials or as temporary materials. These separation processes are sometimes referred to as a release process and may or may not involve the separation of structural material from a build substrate. In many embodiments, sacrificial material within a given build layer is not removed until all build layers making up the three-dimensional structure have been formed. Of course sacrificial material may be, and typically is, removed from above the upper level of a current build layer during planarization operations during the formation of the current build layer. Sacrificial material is typically removed via a chemical etching operation but in some embodiments may be removed via a melting operation or electrochemical etching operation. In typical structures, the removal of the sacrificial material (i.e. release of the structural material from the sacrificial material) does not result in planarized surfaces but instead results in surfaces that are dictated by the boundaries of structural materials located on each build layer. Sacrificial materials are typically distinct from structural materials by having different properties therefrom (e.g. chemical etchability, hardness, melting point, etc.) but in some cases, as noted previously, what would have been a sacrificial material may become a structural material by its actual or effective encapsulation by other structural materials. Similarly, structural materials may be used to form sacrificial structures that are separated from a desired structure during a release process via the sacrificial structures being only attached to sacrificial material or potentially by dissolution of the sacrificial structures themselves using a process that is insufficient to reach structural material that is intended to form part of a desired structure. It should be understood that in some embodiments, small amounts of structural material may be removed, after or during release of sacrificial material. Such small amounts of structural material may have been inadvertently formed due to imperfections in the fabrication process or may result from the proper application of the process but may result in features that are less than optimal (e.g. layers with stairs steps in regions where smooth sloped surfaces are desired. In such cases the volume of structural material removed is typically minuscule compared to the amount that is retained and thus such removal is ignored when labeling materials as sacrificial or structural. Sacrificial materials are typically removed by a dissolution process, or the like, that destroys the geometric configuration of the sacrificial material as it existed on the build layers. In many embodiments, the sacrificial material is a conductive material such as a metal. As will be discussed hereafter, masking materials though typically sacrificial in nature are not termed sacrificial materials herein unless they meet the required definition of sacrificial material.

“Supplemental sacrificial material” as used herein refers to a material that does not form part of the structure when the structure is put to use and is not added as part of the build layers but instead is added to a plurality of layers simultaneously (e.g. via one or more coating operations that applies the material, selectively or in a blanket fashion, to a one or more surfaces of a desired build structure that has been released from an initial sacrificial material. This supplemental sacrificial material will remain in place for a period of time and/or during the performance of certain post layer formation operations, e.g. to protect the structure that was released from a primary sacrificial material, but will be removed prior to putting the structure to use.

“Primary sacrificial material” as used herein is a sacrificial material that is located on a given build layer and which is typically deposited or applied during the formation of that build layer and which makes up more than 20% of the sacrificial material volume of the given build layer. In some embodiments, the primary sacrificial material may be the same on each of a plurality of build layers or may be different on different build layers. In some embodiments, a given primary sacrificial material may be formed from two or more materials by the alloying or diffusion of two or more materials to form a single material.

“Secondary sacrificial material” as used herein is a sacrificial material that is located on a given build layer and is typically deposited or applied during the formation of the build layer but is not a primary sacrificial materials as it individually accounts for only a small volume of the sacrificial material associated with the given layer. A secondary sacrificial material will account for less than 20% of the volume of the sacrificial material associated with the given layer. In some preferred embodiments, each secondary sacrificial material may account for less than 10%, 5%, or even 2% of the volume of the sacrificial material associated with the given layer. Examples of secondary structural materials may include seed layer materials, adhesion layer materials, barrier layer materials (e.g. diffusion barrier material), and the like. These secondary sacrificial materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931. In other embodiments, such coatings may be applied in a non-planar manner, for example, in openings in and over a patterned masking material that has been applied to previously planarized layers of material as taught in U.S. patent application Ser. No. 10/841,383. These referenced applications are incorporated herein by reference as if set forth in full herein.

“Adhesion layer”, “seed layer”, “barrier layer”, and the like refer to coatings of material that are thin in comparison to the layer thickness and thus generally form secondary structural material portions or sacrificial material portions of some layers. Such coatings may be applied uniformly over a previously formed build layer, they may be applied over a portion of a previously formed build layer and over patterned structural or sacrificial material existing on a current (i.e. partially formed) build layer so that a non-planar seed layer results, or they may be selectively applied to only certain locations on a previously formed build layer. In the event such coatings are non-selectively applied, selected portions may be removed (1) prior to depositing either a sacrificial material or structural material as part of a current layer or (2) prior to beginning formation of the next layer or they may remain in place through the layer build up process and then etched away after formation of a plurality of build layers.

“Masking material” is a material that may be used as a tool in the process of forming a build layer but does not form part of that build layer. Masking material is typically a photopolymer or photoresist material or other material that may be readily patterned. Masking material is typically a dielectric. Masking material, though typically sacrificial in nature, is not a sacrificial material as the term is used herein. Masking material is typically applied to a surface during the formation of a build layer for the purpose of allowing selective deposition, etching, or other treatment and is removed either during the process of forming that build layer or immediately after the formation of that build layer.

“Multilayer structures” are structures formed from multiple build layers of deposited or applied materials.

“Multilayer three-dimensional (or 3D or 3-D) structures” are Multilayer Structures that meet at least one of two criteria: (1) the structural material portion of at least two layers of which one has structural material portions that do not overlap structural material portions of the other.

“Complex multilayer three-dimensional (or 3D or 3-D) structures” are multilayer three-dimensional structures formed from at least three layers where a line may be defined that hypothetically extends vertically through at least some portion of the build layers of the structure will extend from structural material through sacrificial material and back through structural material or will extend from sacrificial material through structural material and back through sacrificial material (these might be termed vertically complex multilayer three-dimensional structures). Alternatively, complex multilayer three-dimensional structures may be defined as multilayer three-dimensional structures formed from at least two layers where a line may be defined that hypothetically extends horizontally through at least some portion of a build layer of the structure that will extend from structural material through sacrificial material and back through structural material or will extend from sacrificial material through structural material and back through sacrificial material (these might be termed horizontally complex multilayer three-dimensional structures). Worded another way, in complex multilayer three-dimensional structures, a vertically or horizontally extending hypothetical line will extend from one or structural material or void (when the sacrificial material is removed) to the other of void or structural material and then back to structural material or void as the line is traversed along at least a portion of the line.

“Moderately complex multilayer three-dimensional (or 3D or 3-D) structures are complex multilayer 3D structures for which the alternating of void and structure or structure and void not only exists along one of a vertically or horizontally extending line but along lines extending both vertically and horizontally.

“Highly complex multilayer (or 3D or 3-D) structures are complex multilayer 3D structures for which the structure-to-void-to-structure or void-to-structure-to-void alternating occurs once along the line but occurs a plurality of times along a definable horizontally or vertically extending line.

“Up-facing feature” is an element dictated by the cross-sectional data for a given build layer “n” and a next build layer “n+1” that is to be formed from a given material that exists on the build layer “n” but does not exist on the immediately succeeding build layer “n+1”. For convenience the term “up-facing feature” will apply to such features regardless of the build orientation.

“Down-facing feature” is an element dictated by the cross-sectional data for a given build layer “n” and a preceding build layer “n−1” that is to be formed from a given material that exists on build layer “n” but does not exist on the immediately preceding build layer “n−1”. As with up-facing features, the term “down-facing feature” shall apply to such features regardless of the actual build orientation.

“Continuing region” is the portion of a given build layer “n” that is dictated by the cross-sectional data for the given build layer “n”, a next build layer “n+1” and a preceding build layer “n−1” that is neither up-facing nor down-facing for the build layer “n”.

“Minimum feature size” refers to a necessary or desirable spacing between structural material elements on a given layer that are to remain distinct in the final device configuration. If the minimum feature size is not maintained on a given layer, the fabrication process may result in structural material inadvertently bridging the two structural elements due to masking material failure or failure to appropriately fill voids with sacrificial material during formation of the given layer such that during formation of a subsequent layer structural material inadvertently fills the void. More care during fabrication can lead to a reduction in minimum feature size or a willingness to accept greater losses in productivity can result in a decrease in the minimum feature size. However, during fabrication for a given set of process parameters, inspection diligence, and yield (successful level of production) a minimum design feature size is set in one way or another. The above described minimum feature size may more appropriately be termed minimum feature size of sacrificial material regions. Conversely a minimum feature size for structure material regions (minimum width or length of structural material elements) may be specified. Depending on the fabrication method and order of deposition of structural material and sacrificial material, the two types of minimum feature sizes may be different. In practice, for example, using electrochemical fabrication methods and described herein, the minimum features size on a given layer may be roughly set to a value that approximates the layer thickness used to form the layer and it may be considered the same for both structural and sacrificial material widths and lengths. In some more rigorously implemented processes, examination regiments, and rework requirements, it may be set to an amount that is 80%, 50%, or even 30% of the layer thickness. Other values or methods of setting minimum feature sizes may be set.

Intravascular Ultrasound Devices (IVUS Devices):

In some embodiments, the intravascular ultrasound devices are intended to be ‘forward-looking’; i.e., the image obtained by the device is of structures in front of, or ahead of the distal end of the device.

In a first embodiment, the IVUS device is designed in the form of a catheter whose diameter is in the range of 1-2 mm (3-6 French). Like most conventional “side-looking” IVUS catheters, the device comprises at least one ultrasonic transducer (e.g., a piezoelectric crystal such as PZT), typically operating at a frequency of several tens of MHz, as well as a drive cable or other flexible member which provides mechanical motion of the transducer to scan its focal point across the field of view of the device. The transducer is bi-directional, and can thus both emit ultrasonic waves, as well as detect reflections of these waves, off structures in the vicinity of the catheter, that return to the transducer. The transducer normally alternates between sending and receiving modes, and requires only a single pair of conductors for its electrical connection. In a conventional side-looking device, the active face of the transducer is typically parallel to the longitudinal axis of the drive cable, and rotation of the drive cable scans the focal point in a circular path, to form a cross-sectional image of the blood vessel (or other structure) within which the catheter is placed.

In the present embodiment, the motion of the spinning cable is instead converted by a miniaturized mechanism to a reciprocating motion of the transducer about an axis perpendicular to this longitudinal axis. In this way, the focal point is scanned across the structures ahead of the distal tip of the catheter, such that the device can image ‘where it is going’. Such a forward-looking capability is extremely valuable in helping to navigate through the vasculature, especially in situations where there are bifurcations, occlusions, and other more complex structures. For example, a side-looking IVUS catheter may not be used to image very narrow or total occlusions of the coronary arteries since the aperture is too small to admit the catheter. Use of a forward-looking IVUS catheter can reduce the reliance on fluoroscopy and its associated X-ray exposure when performing various interventions. U.S. Pat. No. 5,373,849 assigned to Cardiovascular Imaging Systems describes a forward-looking catheter with some similarities to that of the present embodiment; however, the device disclosed in this patent is approximately 4 mm in diameter and could not be made small enough for use in the coronary arteries, and would likely be costly to manufacture. This patent is hereby incorporated herein by reference. As mentioned already, the present device can be manufactured using the EFAB technology of Microfabrica Inc. with the device having a diameter in the 1-2 mm range and by wafer-scale batch fabrication techniques that provide monolithic fabrication which minimize the need for assembly. These difference can lead to more economical fabrication as well as a more reliable and useful device.

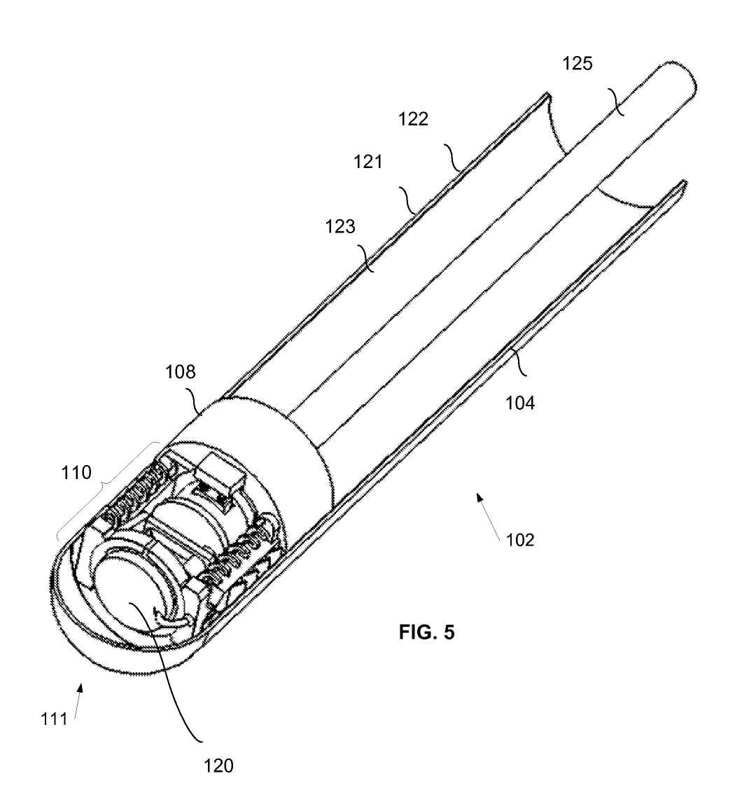

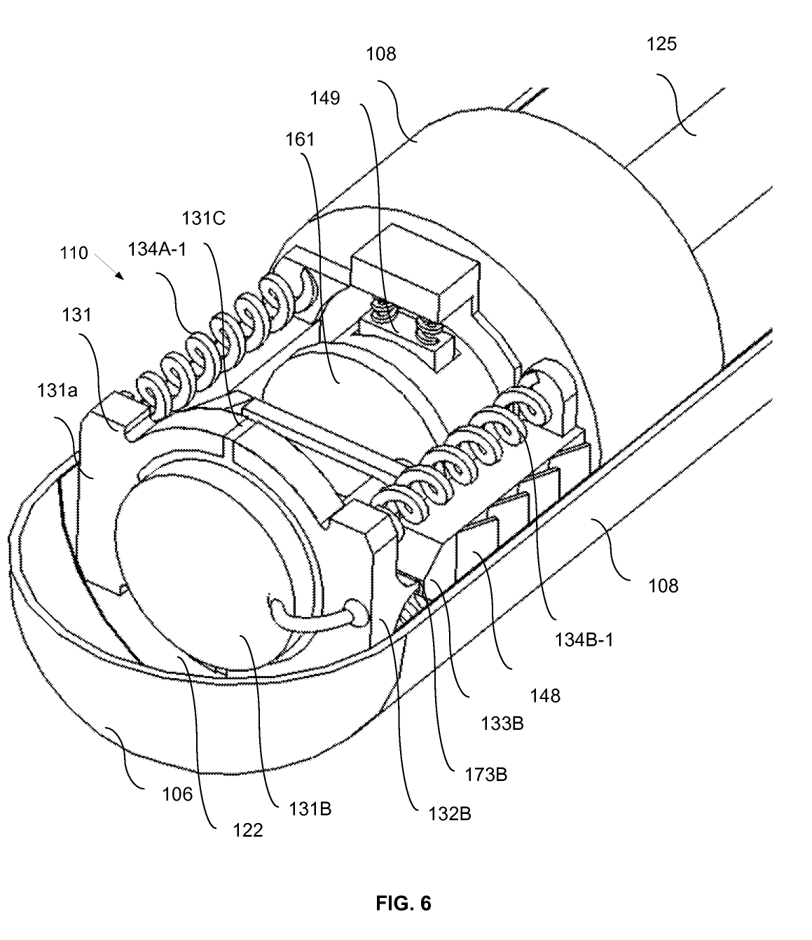

FIG. 5 provides a cut perspective view of the device 102 of the first embodiment. The device 102 comprises a mechanism 110 attached to an insulating (e.g., alumina) substrate 108. The mechanism and substrate are enclosed within a catheter tube 104, preferably having a dome 106 at the distal end 111 which encloses the mechanism 110. Mounted to the distal end of the mechanism 110 is the transducer 120. In this embodiment, the catheter tube 104 has an outer surface 121 that is composed of an insulator and an the inner surface 122 is composed of or lined with an electrically-conductive material 123 that interfaces with a part of the mechanism so as to provide one of two required electrical signal paths to the transducer. Typically coaxial with the catheter tube is a drive cable 125 which passes through the substrate and is fastened at its distal end to the mechanism; rotation of the drive cable, e.g. using a drive motor at its proximal end (not shown) is transmitted to the cable's distal end, causing rotation of a portion of the mechanism 110.

In some alternative embodiments where devices are fabricated using multilayer electrochemical fabrication methods the insulting substrate may be the substrate on which the metal layers are fabricated or in other embodiments, the dielectric substrate may be formed along with the creation of the conductive portions of the devices.

In some alternative embodiments only discrete portions of the inner surface 122 of the catheter 104 need carry the conductive material 123. In still other embodiments the conductive path 123 may alternatively be carried by a wire or other conductive element that extends down the lumen of the catheter, as part of the drive cable or separate therefrom.

In some alternative embodiments, the drive cable may be replaced via a chain like structure, e.g. a chain of universal joints, that allow reliable non-buckling rotation. In other alternative embodiments the universal joints may take the form of joints or flexures that allow certain angular rotations without allowing 360 degrees rotation.

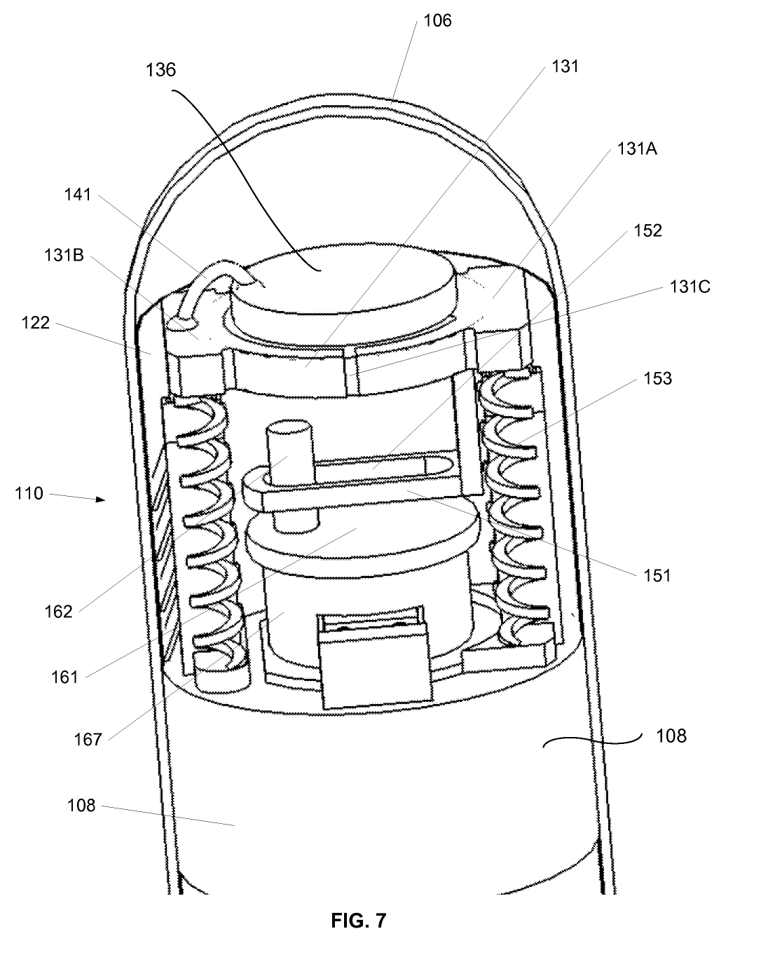

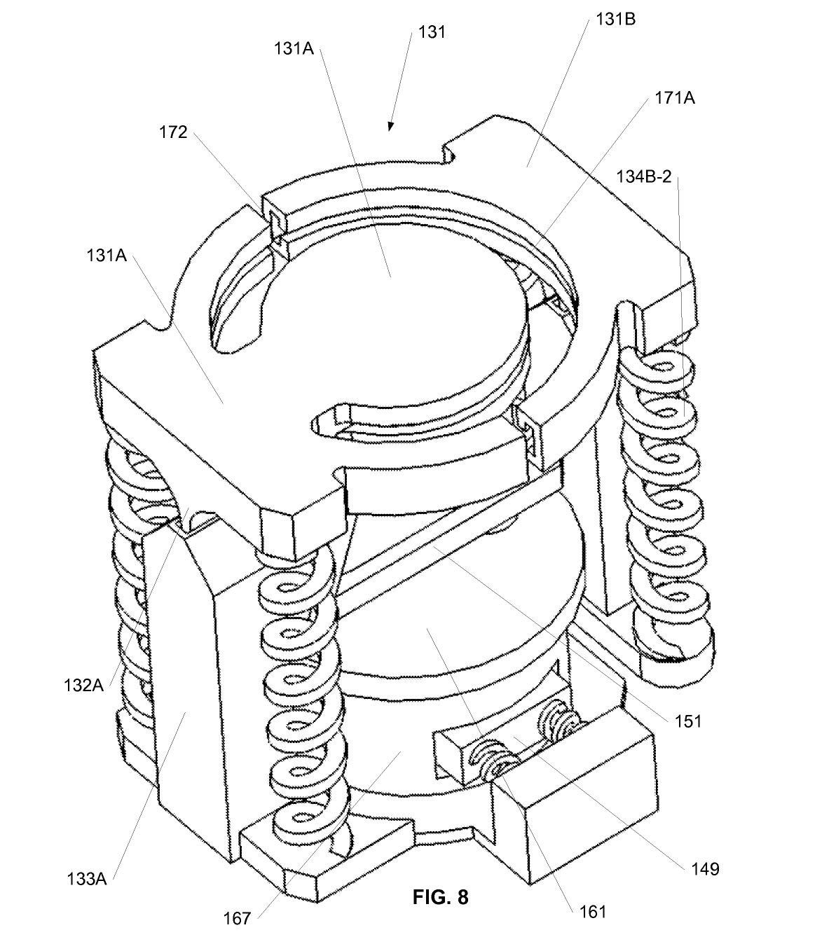

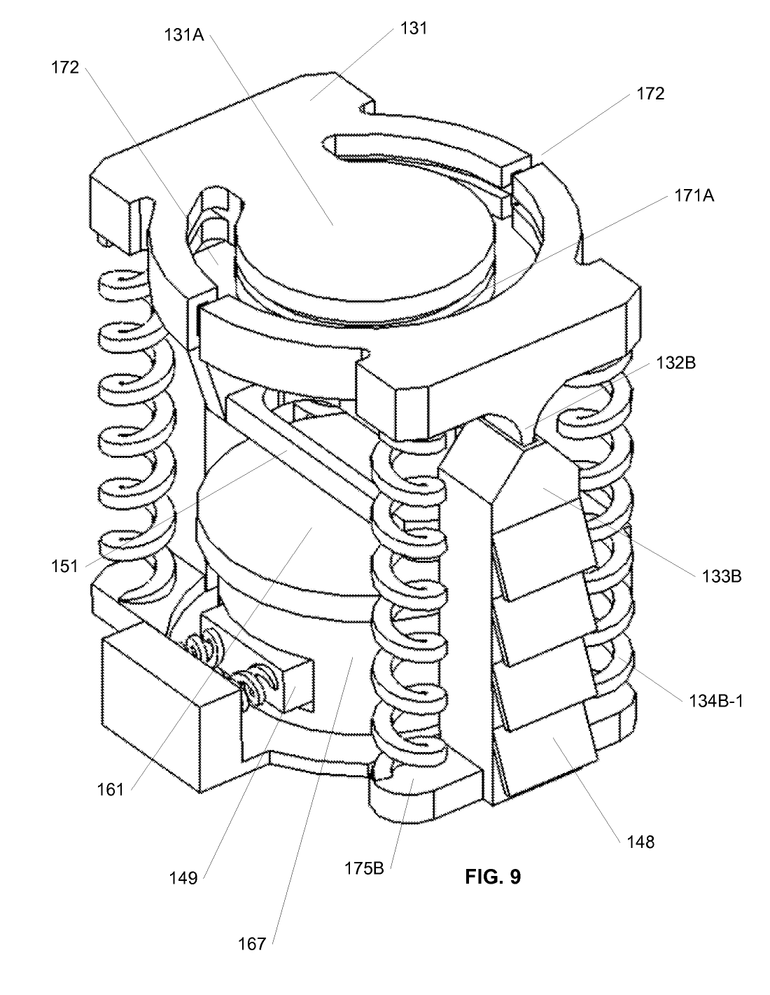

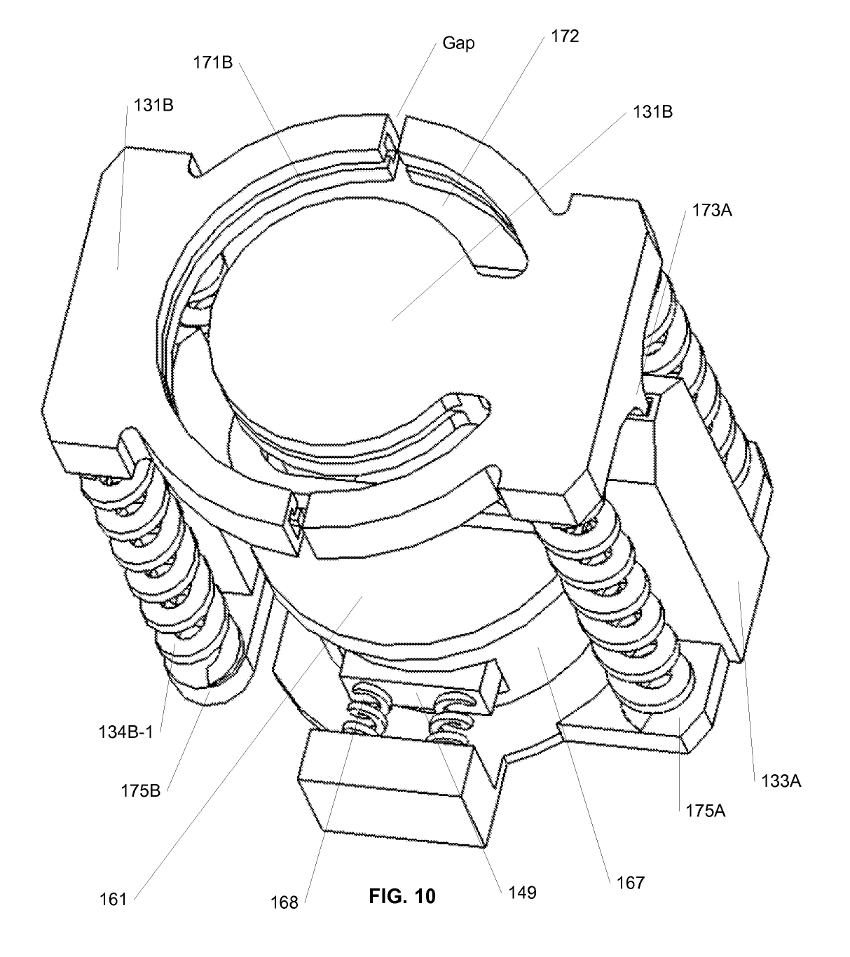

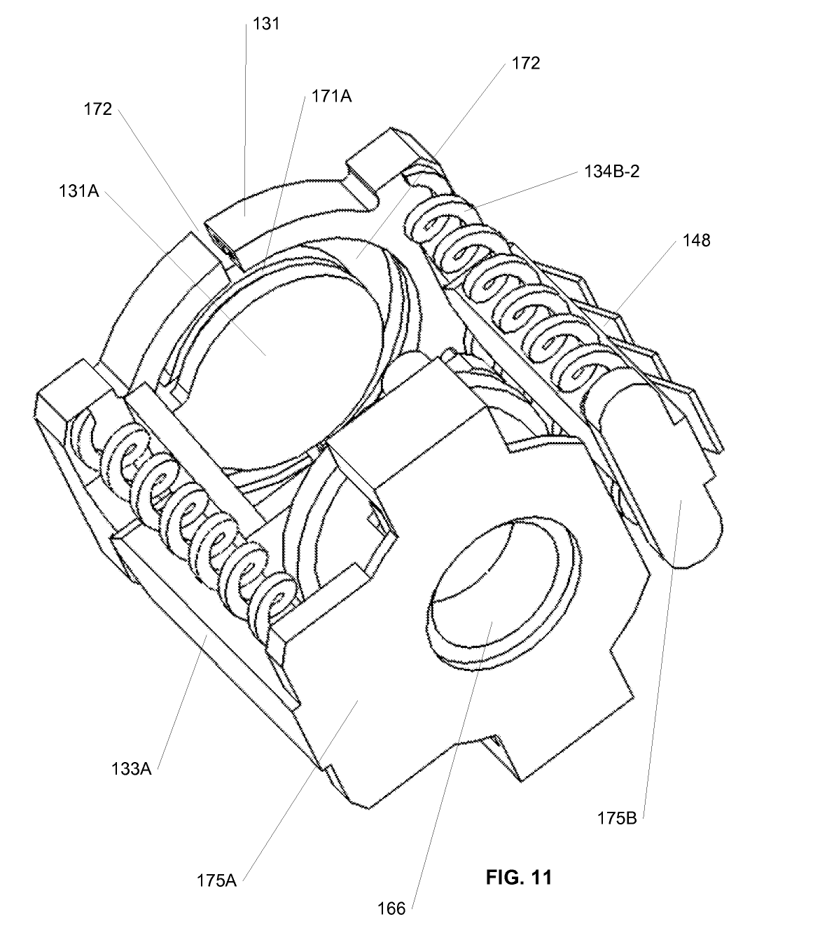

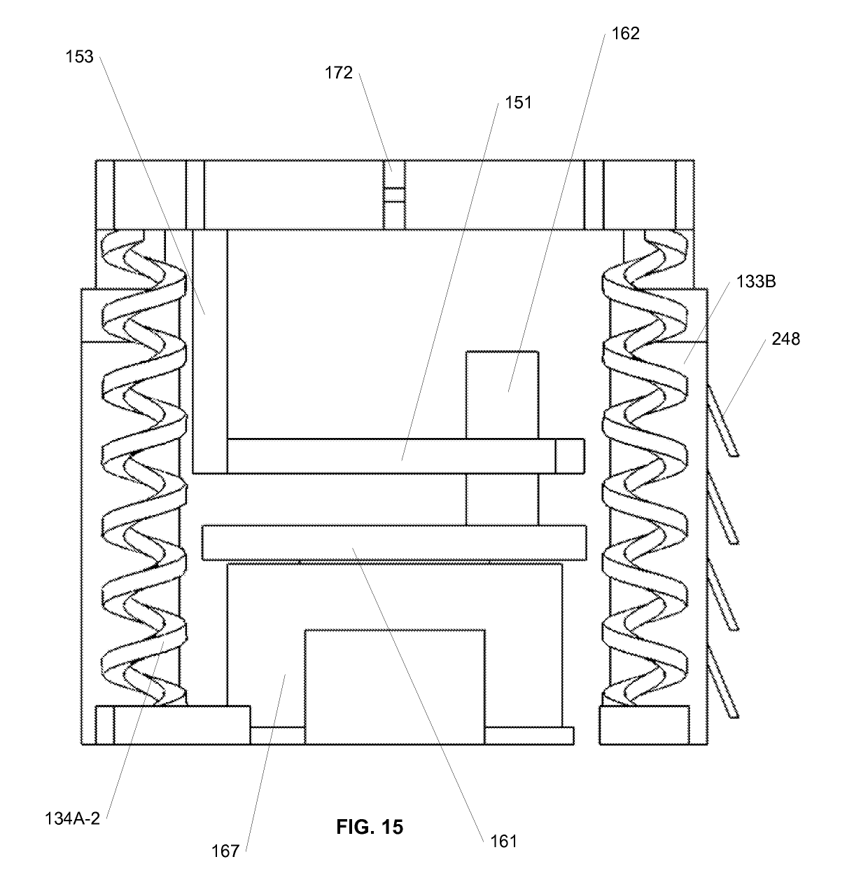

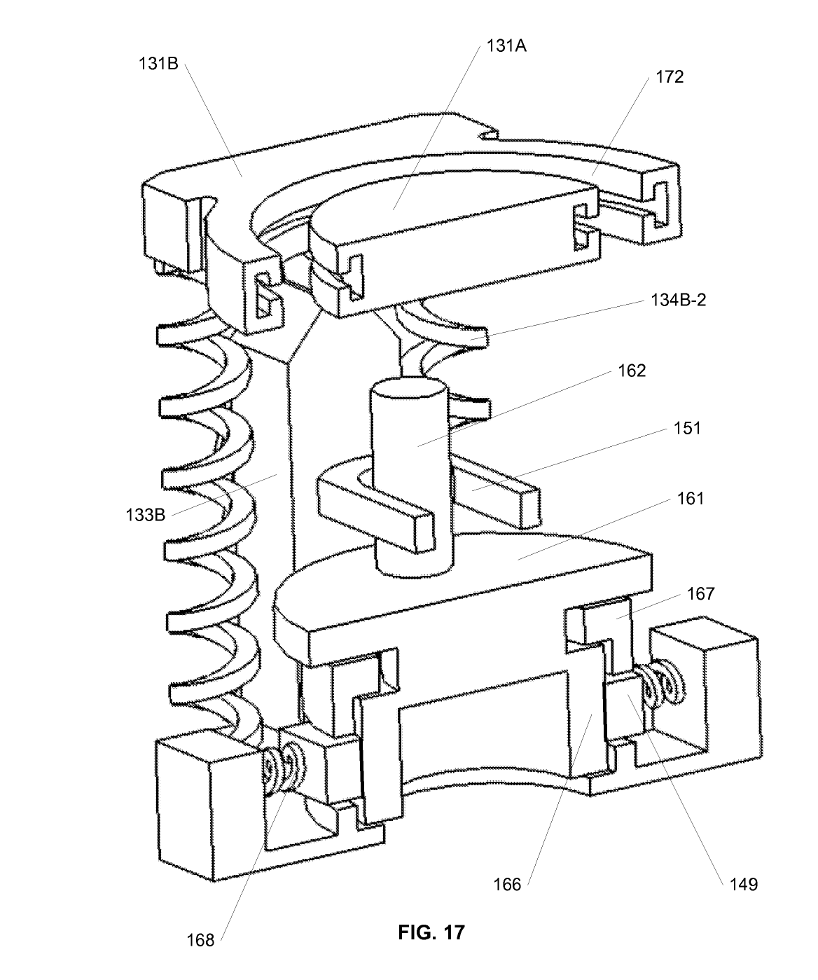

In FIGS. 6-7 the distal end of the device is seen in closer views. FIGS. 8-18 illustrate the mechanism of the device with other components (and the structural dielectric) removed for clarity. The upper portion of the mechanism 110 includes a carriage 131 that can rock from side to side on two fulcrums 132A and 132B that are supported by twin pillars 133A and 133B; the fulcrums are preloaded against the pillars by four carriage springs 134A-1, 134A-2, 134B-1, and 134B-2 which are in tension at all angles that the carriage may assume in normal operation, so that the fulcrums 132A and 132B cannot lift off the pillars 133A and 133B. These springs 134A-1 to 134B-2 also provide reliable/continuous electrical contacts to the moving carriage 131 as it rocks. Rocking of the carriage with its attached transducer 136 causes the transducer to scan its focal point across the field of view. The carriage 131 is divided into two metallic sections 131A and 131B that are joined mechanically, but not electrically, by a structural dielectric 131C (e.g., epoxy); a signal A (e.g., applied voltage) is conducted along one of these sections, 131A, and signal B (e.g., ground) along the other, 131B. The transducer 136, which typically is metallized on both its distal and proximal faces, is mounted to the distal surface of the carriage with a conductive material (e.g., conductive epoxy) so that electrical contact for signal A is made between the bottom of the transducer 136 and one section of the carriage (hereinafter, section 131A). The transducer 136 may (as shown) overlap the metallic portion of section A and thereby extend into the dielectric section of the carriage, as long as it does not make contact with section 131B. The distal surface of the transducer may be electrically connected to the other section 131B of the carriage 131 by conventional means, e.g., using a wire 141, e.g. via wire bonding, as is shown in the figures, to provide the electrical path for signal B. Both mounting of the transducer 136 and wire 141 may be performed at the wafer scale (e.g., using pick and place equipment to place the transducer) for all devices which are fabricated, possibly before the final release of a sacrificial material which is in conjunction with one or more structural material when forming most if not all metallic portions of the mechanism via an electrochemical fabrication process. In some embodiments, rather than bonding the transducer end of the wire 141 to the active surface (i.e. upper, front, or distal surface) of the transducer 136 as shown in the figures, this end may be placed alongside the transducer and the gap between it and the metallized front surface of the transducer 136 may be bridged with solder, by electroplated metal, or the like. One purpose for using such an alternative technique may be to minimize the effect of the wire on the acoustic properties of the transducer. In other alternative embodiments, rather than using a separate piece of wire, an extension of the fabricated structure itself may serve as the wire the distal end of which may be bonded to the transducer in any appropriate manner or electrical contact simply established by elastic force (e.g. spring contact).

On the opposite (more proximal) surface of the carriage is a yoke 151 having a slot 152 which is attached to section 131A through a standoff 153. Proximal to the yoke is a rotating disk 161 having an eccentric drive pin 162 which extends distally from the disk and enters the slot 152 in yoke 151. The disk 161 is attached to the drive cable 125 (passing through a hole in the substrate 108) through a coupler 166, and the latter is supported by a bushing or bearing 167 (which may include a roller or needle bearing in which all the elements are fabricated using, e.g. an electrochemical fabrication process), allowing it to spin. When the disk 161 spins, the pin forces the yoke 151 to move in an oscillatory fashion while the pin moves within the slot 152; the oscillating motion of the yoke 151 rocks the carriage 131 back and forth on the fulcrums 132A and 132B.

Electrical signal paths A is coupled through the drive cable 125. Reliable electrical contact between the drive cable 125 and section 131A is obtained by spring-loaded contact shoes 149 which engage the sides of the coupler 166 through apertures 172 in the bushing 167 and springs 168. This arrangement is similar to the arrangement of brushes in a DC motor. These shoes 149 can be preloaded against the coupler 166 after fabrication (as-fabricated using one of the electrochemical fabrication methods discussed herein there would necessarily be a gap), e.g., by sliding them laterally and holding them in place using a ratcheting or similar mechanism (not shown).

In some alternative embodiments, the yoke 151 and slot 152 may be replaced by a plate while the pin 162 is replaced by an off axis knob or protrusion on the one of the disks that causes pivoting of one plate relative to the other as the protruding element rotates through various angular positions about the axis of the catheter. In other embodiments a single protrusion may be replaced by a plurality of protrusions. In still other embodiments, other mechanical elements may be used to cause tilting upon rotation. The tilting provided by these alternative embodiments may cause a focal point of the system to trace out a scan along a single axis or the focal plane may trace out a two dimensional pattern as the tilting causes a two dimensional tilting of the transducer.

Electrical contact between section 131B of the carriage and the conductive lining of the catheter 122 may be achieved by compliant contact fingers 148 attached to the pillar supporting section 131B. When the device is assembled (which involves inserting the substrate 108 with attached mechanism 110 into the catheter tube 104 of diameter 105), the fingers 148 engage the conductive lining 122, deflecting slightly and providing a continuous contact force against the lining. The fingers are preferably coated with a non-oxidizing material, e.g. gold or rhodium. In some embodiments, the perimeter of the substrate may be trimmed (e.g. using a laser) prior to release of sacrificial material such that the fingers in their undeflected, as-fabricated state can extend beyond its diameter.

FIGS. 8-18 provide further perspective views of the mechanism 110 of the device 102 with various components (and the structural dielectric) removed for clarity. The two sections 131A and 131B of the carriage are seen clearly and it may be noted that they are not in electrical (or, without the dielectric, in mechanical contact with one another). With the dielectric 131C in place, the two sections 131A and 131B move as a single rigid structure. The particular design shown—that of a ring split by a gap 172, surrounding a circular core, provides good mechanical strength and maximizes the amount of metal in the carriage. Most of the surfaces of the two carriage sections 131A and 131B adjacent to the dielectric 131C are provided with groves (e.g. a T-shaped grooves 171A and 171B as shown) which provides excellent mechanical interlocking between the dielectric 131C, which has or is made to have a complementary ridge (e.g. the dielectric may be applied as a liquid during the fabrication of the mechanism and may them solidify or be made to solidify), and the metal components of the carriage. Prior to application of the liquid dielectric in an electrochemical fabrication process, the gap 172 may be formed in a sacrificial material or masking material which was made to temporarily filed the gap and then the dielectric material may be added. During formation of the layers that include dielectric material, adhesion layer and seed layer coating may be applied and removed as appropriate. Such techniques are described in some of the applications incorporated herein by reference.

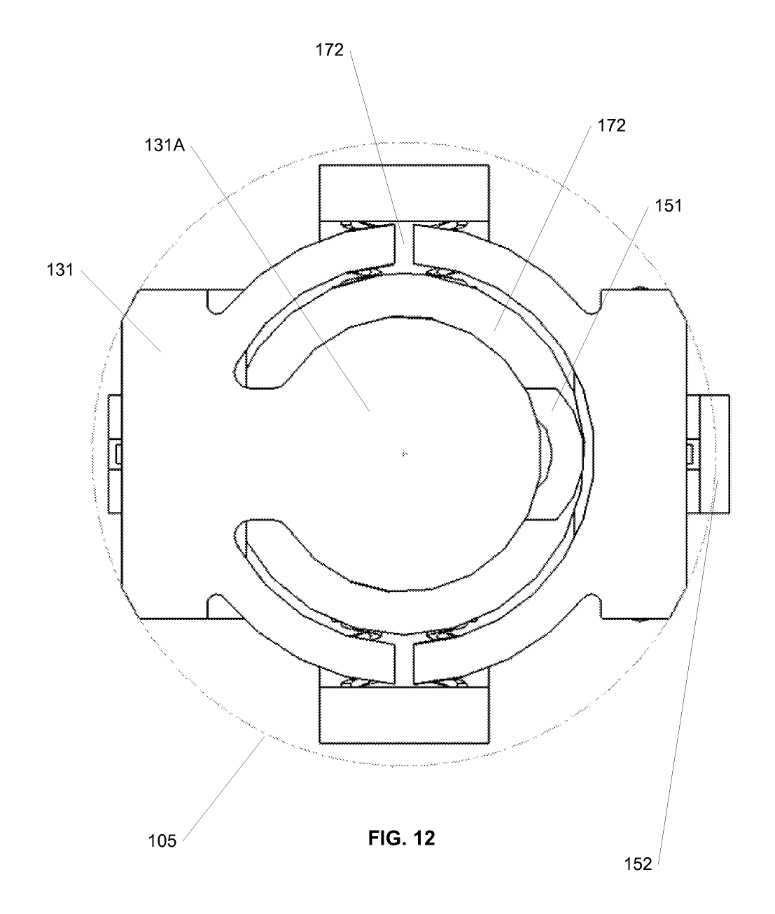

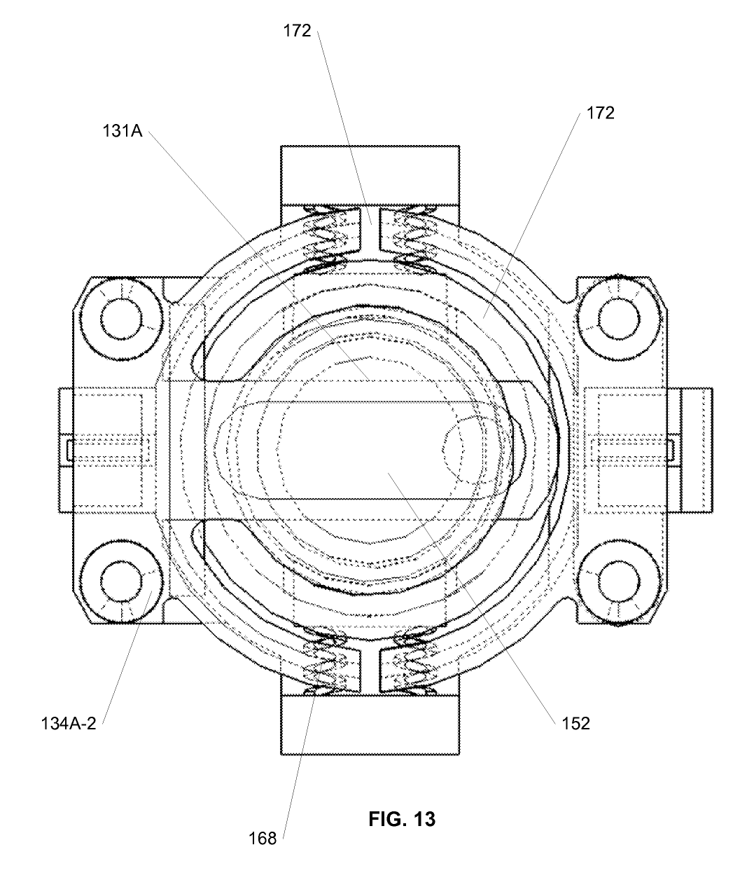

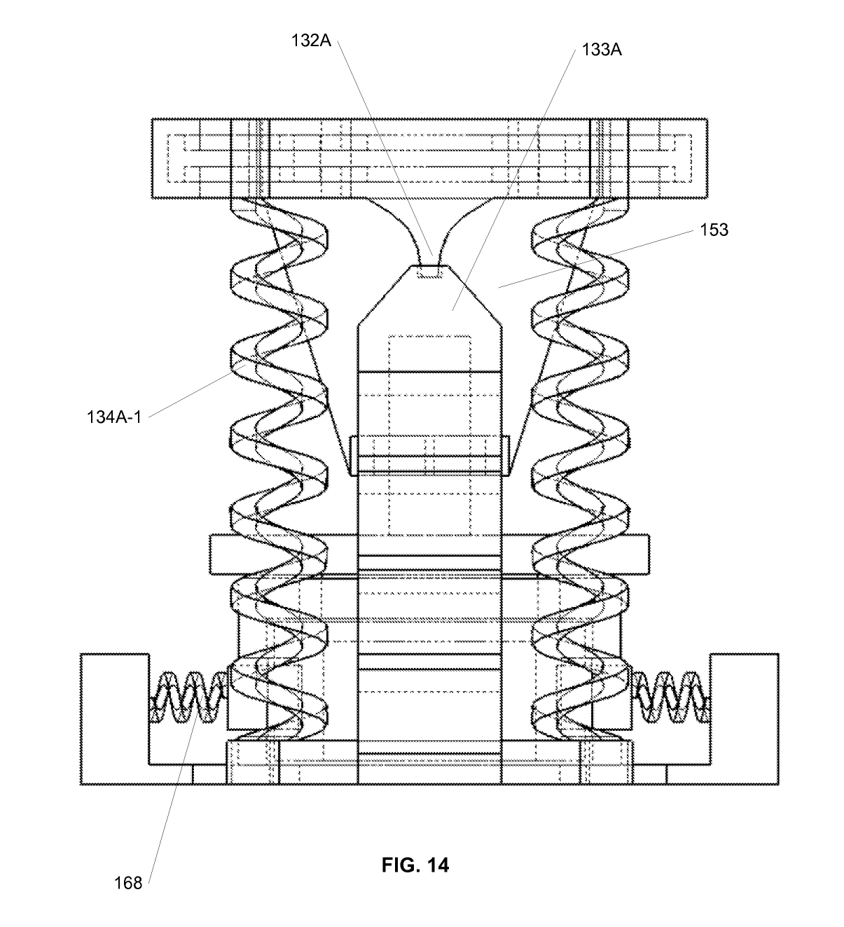

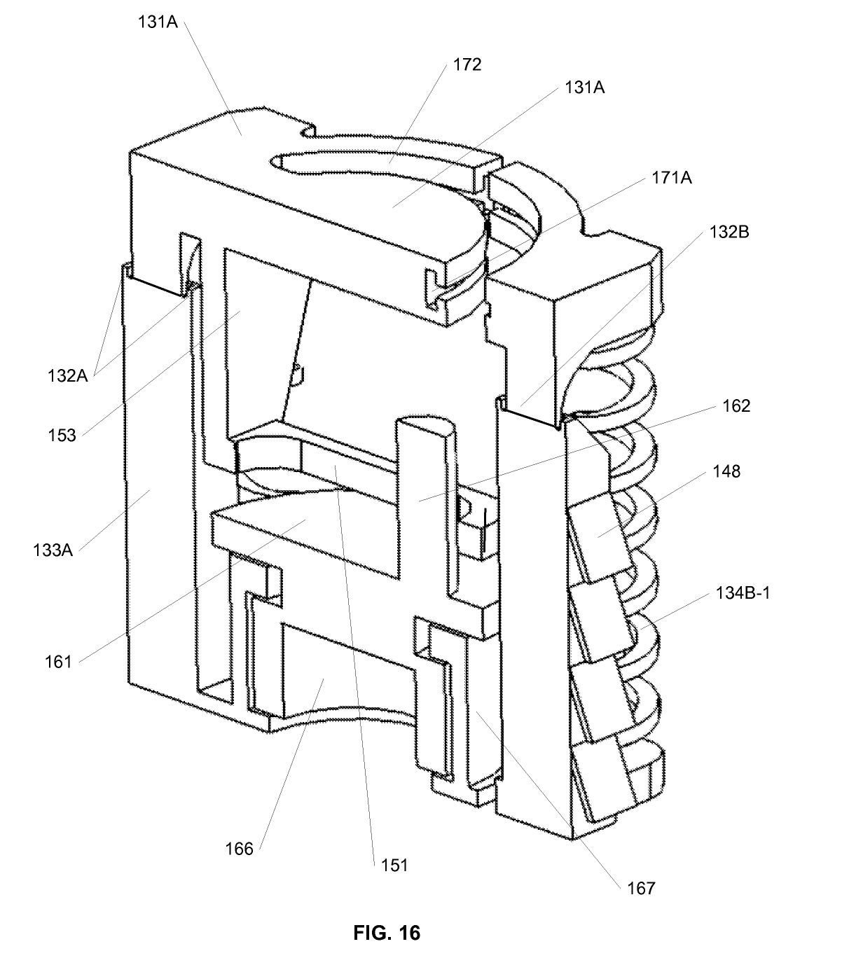

Stops 173A and 173B on all four sides of each fulcrum 133A and 133B prevent the fulcrums 132A and 132B, respectively, from sliding excessively on the pillars; each fulcrum rocks in a trench formed by the stops on top of its pillar. The carriage springs 134A-1 & 134A2 and 134B-1 and 134B-2 extend down to base A, 175A, and base B, 175B, respectively where they attachment to substrate 108 occurs. In a top view (i.e., along the longitudinal axis of the device) such as FIG. 12, the entire device, with the exception of fingers 148 is shown as fitting within a particular diameter 105, which is somewhat smaller than the ID of the conductive lining. The contact fingers extend beyond this circle to contact the conductive lining 122. FIGS. 13-14 show transparent views, while FIG. 16-18 illustrate sectional views, along various planes, of the mechanism.

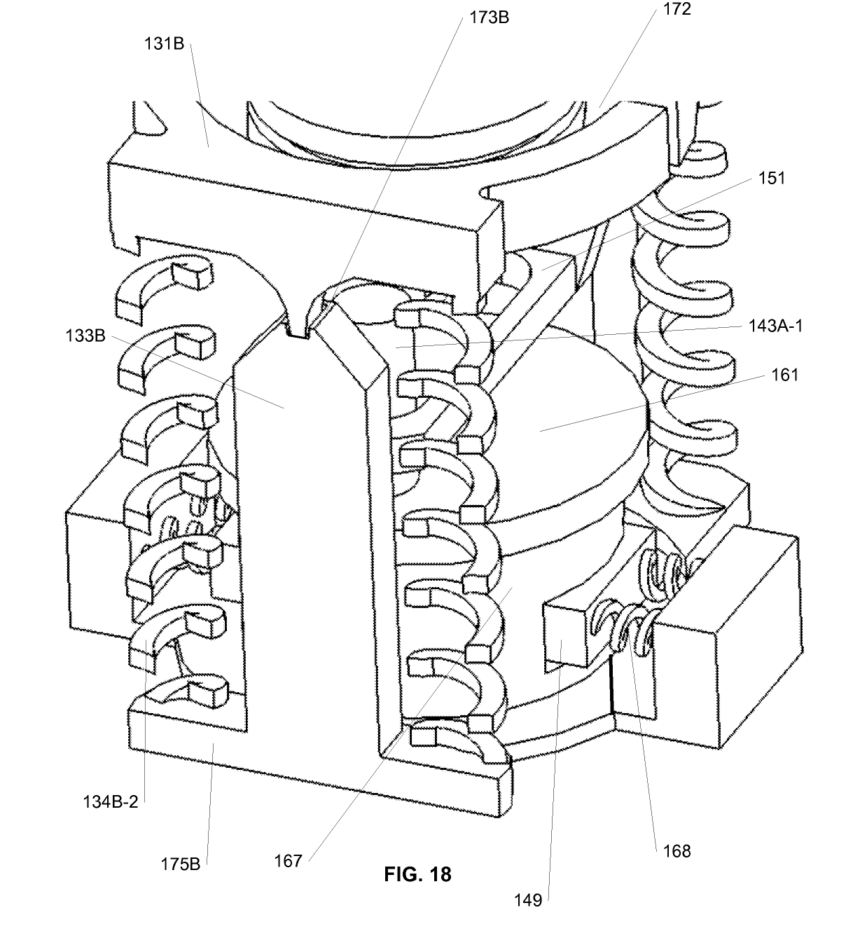

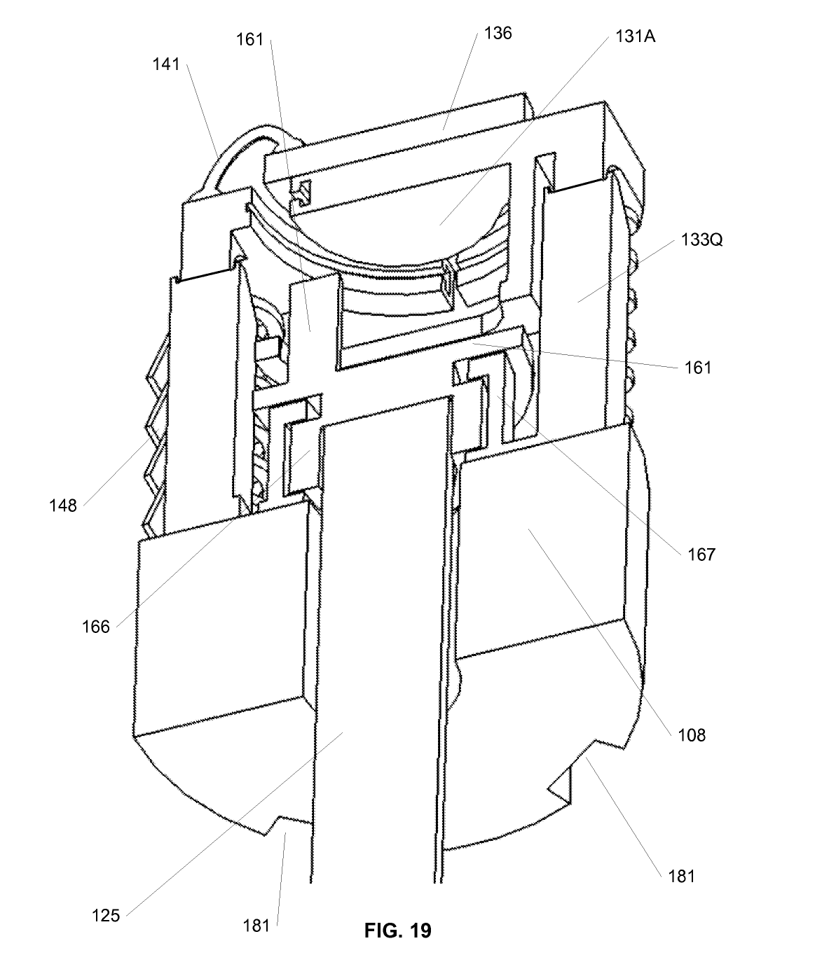

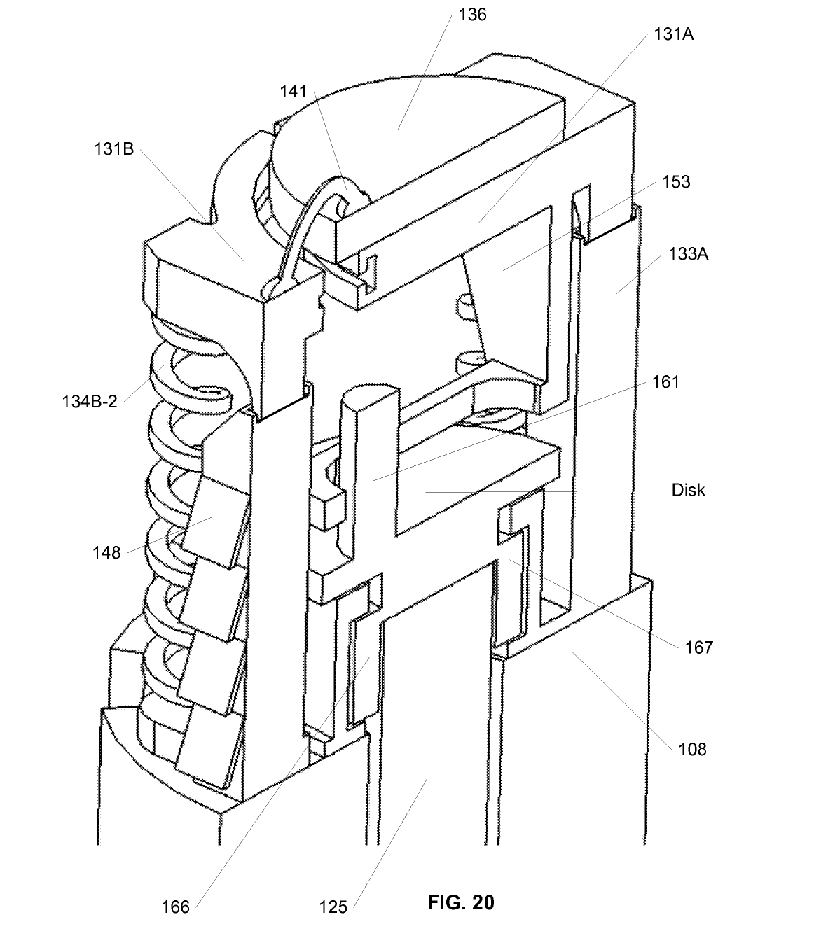

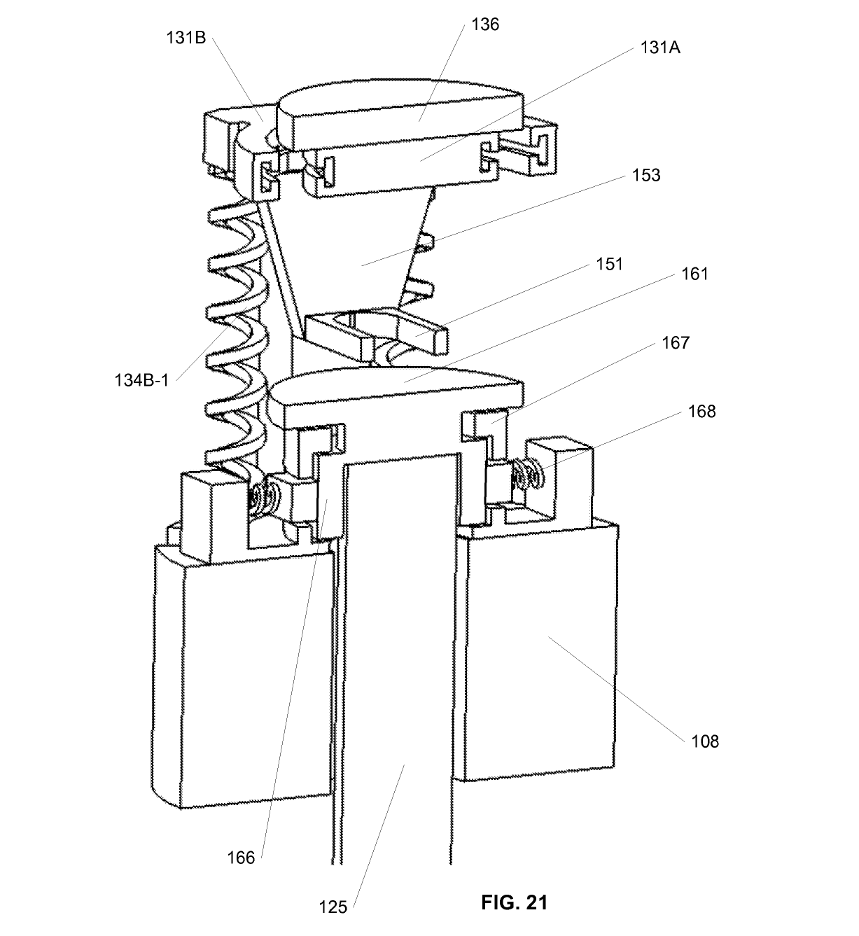

FIGS. 19-21 provide perspective sectional views, along various planes, of the mechanism with drive cable 125, transducer 136, and wire 141 also shown. The drive cable 125 may be attached to the drive cable coupler 166 by various methods including bonding using a conductive adhesive, soldering, brazing, welding, and crimping. To provide proper coupling of ultrasonic waves from the surface of the transducer to the fluid (e.g., blood) outside the catheter, the transducer should be in contact with a coupling liquid (not shown). In the embodiment shown, the coupling liquid should be a dielectric (e.g., an oil) to avoid shorting together paths for signals A and B; however, other embodiments may allow for conductive liquids such as saline to be employed, e.g. by coating various electrically active elements with a dielectric coating material. During the assembly of the device, the substrate 108 and attached mechanism 110 (with transducer already mounted and electrically connected) would typically be slid into the proximal end of the catheter tube 104 toward the distal end and fastened there (e.g., by melting and shrinking an appropriate portion of the catheter tube using localized heating). Notches 181 in the outer diameter of the substrate (shown only if FIG. 19) may be provided to allow coupling fluid to be introduced into the space distal to the substrate after assembly and for air bubbles to be removed from the device.

Since the distal surface of the transducer 136 in the embodiment shown is not coincidence with the rotational axis of the carriage 131 (the latter is approximately coincident with the tips of the fulcrums), the transducer 136 does not only execute a rotation, but also a translation, as it rocks back and forth. In some embodiments, the fulcrums may be located more distally on the carriage such that the amount of translation is minimized or eliminated. The length of the standoff and the amount of eccentricity of the drive pin, among other parameters, may be adjusted to control the field of view (i.e., the angle over which the focal point is scanned).

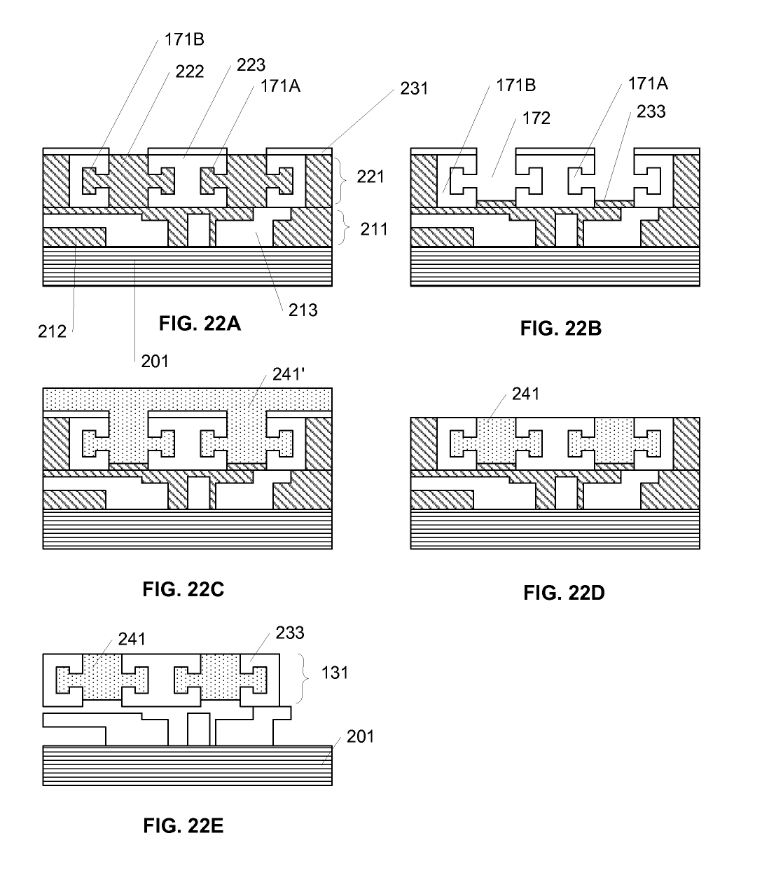

FIGS. 22A-22E depict various states in an example process according to which dielectric structural material may be incorporated into a device produced using an electrochemical fabrication technology, and in particular, they illustrate how a dielectric structural material may form a portion of the carriage 131 as described above. In FIG. 22A, a substrate 201 is shown on which several (random) layers 211 have been formed. Each of these layers are substantially planar and include at least two materials each, e.g. at least one sacrificial material 212 and at least one structural material 213. Above these layers 211, multilayer structure 221 is formed. Structure 221 is similar to that of carriage 131 (with its groove structure 171A and 171B) and is formed of structural material 223 (e.g., they may be nickel cobalt) and is embedded within sacrificial material 221. In different embodiments structural materials 213 and 213 may be the same or different and sacrificial materials 212 and 222 may be the same or different (e.g., the may be copper). Even structural material 223 may actually be a plurality of different materials on different portions of single layers or on different layers. On top of the last layer is a patterned mask 231, which is here assumed to be composed of structural material but may be a photoresist or other material in alternative embodiments.

In FIG. 22B, the structure has been partially released (i.e. the sacrificial material has been partially removed). The partial release has completely exposed the grooves 171A and 171B and the intervening 172 without removing significant sacrificial material from below the carriage. Etching has occurred selectively through mask 231. This partial release would typically be achieved through a timed etch resulting in sacrificial material approximately below the level of the bottom of the carriage not being removed (in the illustration of FIG. 22B, the etch is stopped somewhat above this level). The resulting top surface 233 of the remaining sacrificial material will in general define the bottom surface of the dielectric material to be added. At this point, the mask may be removed if desired, however, in the embodiment shown, it is removed as part of a later planarization step.

In FIG. 22C, dielectric precursor material 241′ (e.g., epoxy, glass, thermoplastic, etc.) has been applied in liquid or semi-solid form, backfilling the void left behind by the partial release, and typically extending beyond the mask surface (however, use of doctor blade, squeegee, air knife, or similar device or technique can minimize or completely remove this overburden of material). The dielectric precursor material 241′ has been cured or hardened to create dielectric material 241.

In FIG. 22D, the surface has been planarized to eliminate excess material and (in the case shown), the mask 231.

Finally, in FIG. 22E, the remaining sacrificial material has been removed, showing the final metal/dielectric structure. It will be noted that in this embodiment no further layers are added (e.g., the top of the carriage is the last layer to be fabricated). Additional layers may nonetheless be added in alternative embodiments; however, metallization of the dielectric surface may be required to provide sufficient conductivity for further electroplating, depending on the geometry of the previously formed layers possibly on the geometry of the subsequent layers to be formed.

Of course in other embodiments, the incorporations of a dielectric material may occur via other processes, such as the layer by layer formation and build up of the sacrificial material along with the formation of individual layers of conductive structural material and sacrificial material. In some alternative embodiments, an initial layer of a barrier material (e.g. a dielectric material) may have been used to form an etch barrier so that the etching of the sacrificial material from gap 172 and grooves 173A and 173B would have a better defined end point. After removal of the desired sacrificial material, the initial barrier material could be removed or remain in place. In any event, if a seed layer material remains above the barrier material it must be removed prior to the filling with material 141′.

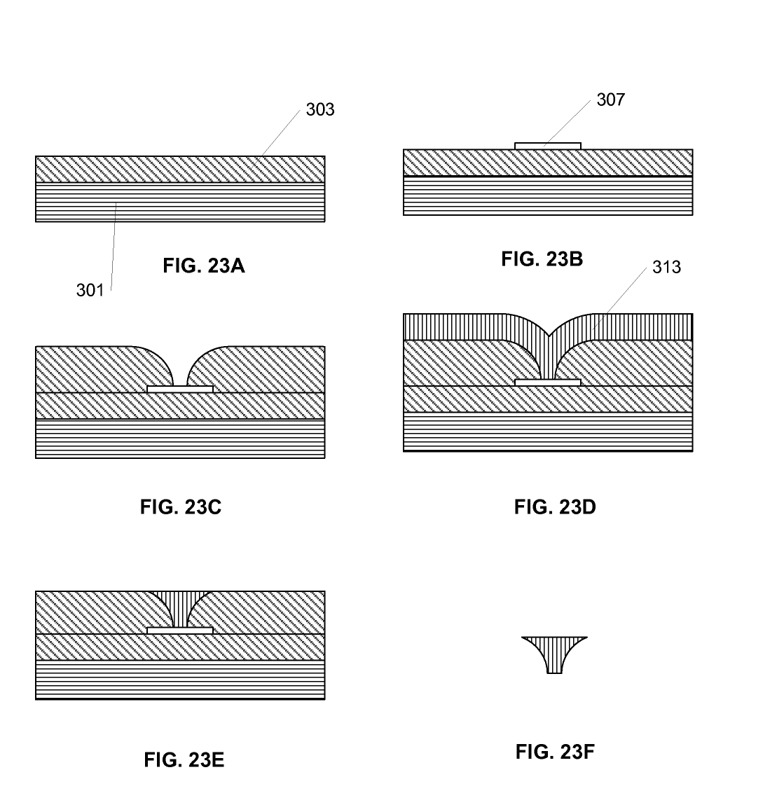

FIG. 23A-33F illustrate various states of an electrochemical fabrication process or forming the fulcrum portions of the devices of FIGS. 5-21. The fulcrum point of each segment of the carriage should be relatively narrow to avoid toggling of the carriage as it rocks back and forth. The fulcrum for each segment of the carriage is preferably elongated to provide a stable attachment to the carriage sections; thus a roughly prismatic shape is indicated. The fulcrum point as well as the distal end of the pillars on which the fulcrums rest are preferably formed form relative hard materials so as to avoid any undesired wear of the two surfaces. The method proposed herein involves a ‘mushrooming’ technique similar to that which may be used to create tips on probes for semiconductor testing as described in U.S. patent application Ser. No. 11/177,798, filed Jul. 5, 2005 by Kim et al. and entitled “Microprobe Tips and Method for Making”. This referenced patent application, as well as all other patent applications referenced herein, is incorporated herein by reference as if set forth in full.

In FIG. 23A, a substrate 301 is shown along with one or more layers of sacrificial material 303 located thereon. In FIG. 23B, photoresist 307 has been patterned in the form of a narrow strip (the long axis of the strip is perpendicular to the plane of the figure). In FIG. 23C, sacrificial material 304 has been plated, mushrooming over the photoresist to form a roughly prismatic mold as designed. Then (not shown) a seed layer (e.g., Cu) and if necessary, an adhesion layer (e.g., Ti) may be deposited on the surface in order to make the exposed photoresist pattern 311 conductive and capable of being plated onto. In FIG. 23D, a suitably wear-resistant structural material 313 (e.g., rhodium, or rhodium backfilled with another material, e.g. nickel or nickel cobalt, or the like) has been deposited into the mold. In FIG. 23E, the surface has been planarized, and in FIG. 23F, the sacrificial material 304, 303, and any seed layer material and adhesion layer material has been removed, releasing the fulcrum. Because the sacrificial material mushrooms in two axes, not just the one as shown in the cross-section of FIG. 23C, the sidewalls of the fulcrums at each end will actually also be curved (not shown in the figures).

In some alternative embodiments, it may not be necessary to use a seed layer material after depositing sacrificial material 304 particularly when the width of the exposed photoresist 311 is very narrow.

In some alternative embodiments, fulcrums would not be formed as individual elements as indicated in this process but this process would be incorporated into a multilayer build process (e.g. as an intermediate layer or portion of an intermediate layer). If the fulcrums are formed as individual elements they may be transferred to, and possibly bonded to structural material existing on previously formed layers or they may be placed and then built upon by subsequently formed layers.

In order to fabricate the fulcrums as described above, and in order to apply tension on the carriage springs, the mechanism may be fabricated with the fulcrums built above (i.e., at a higher layer) than the tops of the pillars, and the proximal (bottom) ends of the carriage springs built above the bases to which they are attached. In other words, certain structures are fabricated and displaced vertically (along the layer stacking axis, e.g. the z-axis) from their final, assembled positions, but may nevertheless be in their correct X/Y positions; i.e., they may be pre-aligned to facilitate assembly. In some embodiments, the proximal spring ends are provided with pads (not shown) coated with an adhesive material (e.g., solder). After release of the entire mechanism, pressure is applied (along the longitudinal axis of the device) to the proximal ends of the springs to press the ends against bases 175A and 175B while bonding them there, stretching the springs (at the same time, the fulcrums are forced up against the tops of the pillars). This assembly process may be performed on a wafer scale, i.e., simultaneously for all mechanisms fabricated together on the EFAB wafer. In some embodiments, temporary fixtures (not shown) may be co-fabricated along with the mechanisms so as to deliver pressure to the pads at the proximal ends of the springs when a plate is pressed against the top of the wafer, for example. Other methods of wafer-scale or individual assembly may be also employed.

In some embodiments, in lieu of a fulcrum with springs, bushings or bearings are provided. This may however introduce complications with the multi-layer electrochemical fabrication of the devices since fabricating the bushings/bearings along the layer stacking axis in a way that allows smooth rotation can be uneconomical, requiring many thin layers to minimize the stairsteps associated with the layer stacking process or use of stair step reduction methods as described in previous patent filings by the same assignee. In some embodiments, a workaround to this is to fabricate the bushings or bearings perpendicular to their final orientation, and then rotate them by 90° through an assembly process.

If desired, the carriage may be designed to articulate with respect to the yoke through a compliant coupling such that only a small motion of the yoke is required to obtain a relatively large angular deflection of the carriage, especially if the latter is operated at a resonant frequency. In general, the mechanism may be operated at the resonant frequency of the carriage or another frequency if resonant behavior is undesirable.

Other means of packaging the device than those described above, and in particular means of achieving electrical contact between sections A and B and the proximal end of the catheter, may be used. For example, in some embodiments, the substrate may be a multi-layer ceramic such as LTCC (or other substrate through which electrical contact can be made between the distal and proximal surfaces through vias). In this case electrical signals applied to the proximal surface of the substrate may be connected through bases A and B, through the carriage springs, and ultimately reach the transducer.

As described above, a laser may be used to cut or trim the substrate. The same laser may also be used to perforate the substrate to allow passage of the drive cable, and to cut the notches. In general, lasers, abrasive jets, water jet-guided lasers, etc. are suitable tools for obtaining the required substrate shape. Ultrasonic machining may also be employed, e.g., to perforate the substrate.

In some embodiments, a second transducer may be incorporated in the device. The second transducer may be oriented conventionally and appropriate electrical connection made such that the resulting device combines both side-looking and forward-looking imaging in same device.

In some embodiments, the yoke may be split, with part of it in contact with section B of the carriage, such that the A and B signal lines are shorted, reversed in polarity, etc. at least once per rotation of the drive cable, to provide a synchronization/encoder pulse to the electronics so that exact angular orientation of the device may be determined. Other methods and electromechanical or optical means for ascertaining angular orientation are also possible and will be understood by those of skill in the art upon review of the teachings herein.

As described, the device is capable of obtaining a 2-D image. The focal spot is moved back and forth along a line and reflections from surrounding structures are received by the transducer and converted into electrical signals. The scan line is associated with one axis of the 2-D image, and the distance to structures (typically as measured by the time of flight for the return pulse) is associated with the other axis. To provide a 3-D image of the surroundings, the scan line must itself be moved. In some embodiments, it may be rotated about the longitudinal axis of the device, creating a radial scan pattern resembling an starburst; this can be done by twisting the entire device about the longitudinal axis at the desired speed. Through appropriate gearing (e.g., a planetary-type gear arrangement) the mechanism may be designed to rotate about the longitudinal axis when driven by the drive cable, without rotation of the entire catheter, preferably in a synchronous manner in which the rotation and the rocking are maintained in a desired relative phase. In some embodiments, rather than twisting the entire device slowly with respect to the rocking motion of the carriage to create a radial scan pattern, the inverse is done. The device is instead twisted rapidly (e.g., 1800 R.P.M. is a common speed for IVUS catheters) and the carriage is rocked relatively slowly (e.g., actuated either by a separate drive cable spinning at a somewhat lower or higher R.P.M.). This creates a scan in the form of concentric circles. In some embodiments, the scan line may be displaced perpendicular to its axis at a relatively slow speed, to form a roughly-rectangular raster scan pattern. In some embodiments, other 2-D scan patterns may be used, including helical and spherical scans.

In some embodiments, in lieu of a single transducer mounted to the carriage, a linear array of transducers may be provided, with the transducers spaced out along a line substantially parallel to the rotational axis of the carriage. If appropriate electrical connections are made to these transducers and appropriate signals applied, the direction of the ultrasonic beam that is emitted from the array may be varied by varying the phase and/or amplitude of the applied signals. With the carriage stationary, purely electronic scanning of the beam along a line can be achieved, and since the scanning is done electronically, it can be very fast. By rocking the carriage at a relatively slow speed using the mechanism described herein, the scan line may be moved substantially perpendicular to itself such that an entire 3-D volume may be raster scanned by the device.

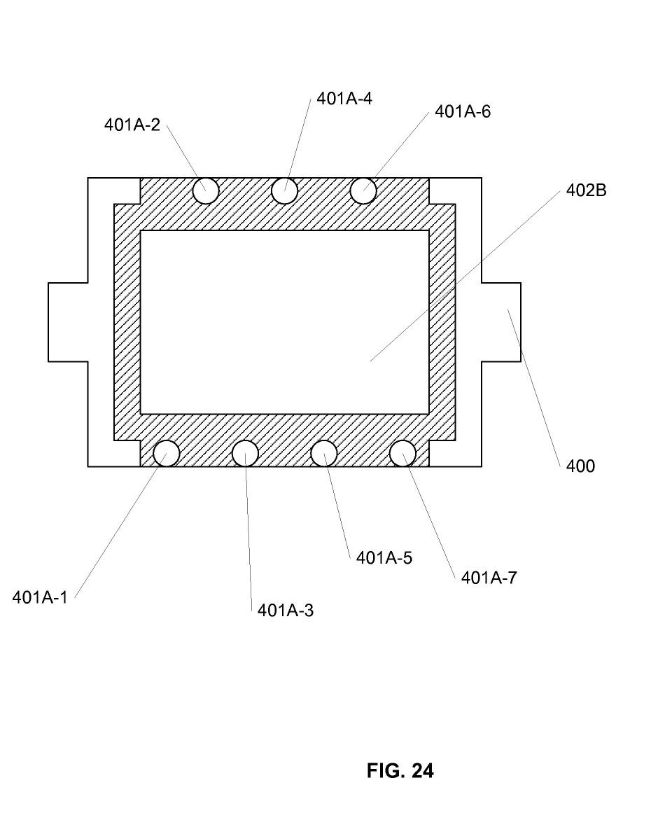

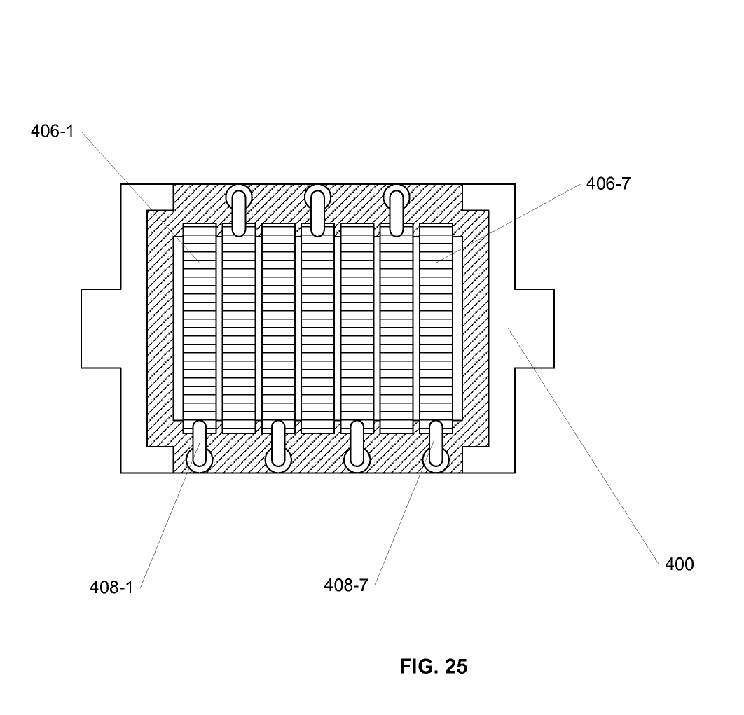

FIG. 24 provides a top view of a carriage 400 having transducer conductors 402A1-402A7 and 402B isolated by dielectric material 404; the carriage accommodates multiple transducers (seven are set forth in this example) and provides an isolated electrical connection (signals A1, A2, etc.) to each of conductors 402A-1 to 402A-7 long with a common signal B applied to conductor 402B (e.g., ground path). The elements may be provided with grooves on their edges, similar to that described above with regard to the embodiment of FIGS. 5-21 to help anchor them to the dielectric 404. Associated with (e.g., underneath) each element is at least one compliant interconnect (e.g., a spring), not shown, isolated electrically from the others. These interconnects may simply be additional, individually-isolated carriage springs which serve a mechanical role as well as the electrical role of carrying signals from the moving carriage to a stationary part of the device. Alternatively, they may be purely electrical interconnects, in which case they may be designed for minimal stiffness. FIG. 25 shows the carriage with the transducers 406-1 to 406-7 mounted to it and electrical connections made via wires 408-1 to 408-7.

In some alternative embodiments instead of using a grid of linear transducers to provide single axis scanning with the array held stationary, it may be possible to produce a two dimensional array of transducers to provide multiple axis scanning with the array held stationary. Such devices exist for scanning electromagnetic signals in the form of butler matrices as described in U.S. patent application Ser. No. 10/607,931 which is incorporated herein by reference as if set forth in full.

In some embodiments, in lieu of a metal/dielectric carriage as described herein, a carriage that incorporates a pre-fabricated interconnect element such as a multi-layer ceramic element (e.g., low-temperature cofired ceramic (LTCC)), a ceramic or silicon element with metallized vias, or a multi-layer organic substrate (e.g., printed circuit board) may be used. This is particularly helpful when there are multiple signals to be routed, as in the case of an array of multiple transducers. In these embodiments, the device may be fabricated upside-down using EFAB technology on top of the interconnect element (using it as a substrate) and the perforated substrate shown in the figures can be bonded to it, either before or after release of sacrificial material (the hole in the latter substrate can assist with the release process if release is done at the wafer scale). Alternatively, the device may be fabricated right side-up as already described, and the interconnect element bonded to it to form at least a portion of the carriage. In either case, bonding can be performed using adhesive, solder, brazing, welding, and other methods known to the art. It may be desirable or even necessary prior to bonding either a substrate or an interconnect element to first stretch the carriage springs (e.g., using the fixture described and bonding the pads to the substrate) and remove any fixturing.

In some embodiments, rather than use a bulk piezoelectric material as the transducer and then mounting and bonding it to the device, the transducer may be formed in an integrated fashion by depositing active material (e.g., a piezoelectric material such as PZT, zinc oxide, etc.) as part of the EFAB process, for example, by sputtering, a sol-gel process, etc. In some embodiments, in lieu of a piezoelectric material, ultrasonic waves may be generated and sensed using vibrating structures (e.g., membranes) relying on electrostatic or electromagnetic actuation and sensing.

In some embodiments, other forms of radiation (other than ultrasound), or other forms of radiation in addition to ultrasound, can be used for imaging in a forward-looking fashion. Examples include catheters for optical coherence tomography (OCT), near-IR imaging catheters that can see through blood, and scanners of light sources and/or detectors, e.g., for optical imaging using visible light. An example of the latter is scanning for a laser scanning camera (e.g., that of Microvision, Inc., Redmond, Wash.). Indeed, a variety of scanning applications may benefit from a scanner of the type described herein, in which what is scanned is a transducer, a light source (e.g., a laser), a mirror, or other device.

In some embodiments, in lieu of the carriage rotating around a fulcrum as described herein, the carriage may rotate on pivots, bushings, or bearings. Such elements may be fabricated at 90° to their final orientation to allow for smooth surfaces which are easy to create in the plane of the layer but more difficult to create along the layer-stacking axis. The elements can be attached to hinges or bendable elements, then rotated into their final positions and locked in place. If such elements are used, then the carriage springs may play less of a role and may in some cases be eliminated if some other electrical interconnect to the moving carriage (flexible element, slip ring, etc.) is provided.

In some alternative devices, where the tilting scan of the transducer occurs along a single line, the fulcrum elements may be moved from a plane that lies along a diagonal of the transducer element to a plane that is closer to one edge of the transducer, a stop added, and the spring count may be reduced from four to two or even to one. The spring or springs may still be operated in a state of tension and the transducer may be mounted at an angle relative to the plane of the layers such that when the spring swings from a tensional minimum to a tensional maximum the transducer swings from one extreme to the other extreme. In still other embodiments, additional spring elements may be added. In some alternative embodiments, IVUS devices of either of the forward-looking type or of the side-looking type may use a fluid driven turbine to spin the distal end of the device as opposed to spinning the entire shaft. The distal end of the device, for example, may be spun by a flow of saline, or the like, that is directed onto the turbine from a tube or channel that extends along the length of the catheter. The drive fluid may be allowed to remain in the body of the patient or it may be extracted via a recovery tube that also extends along the length of the catheter.

In still further embodiments, the springs supporting the carriage may be made to undergo compression as opposed to tension during the scanning of the carriage. In such embodiments, the device may be formed with carriage separated from the substrate by a greater distance than it will have after assembly. After formation of the structure and release of it from sacrificial material, the carriage and substrate may be brought into a more proximal relationship which may be fixed in position by mechanical locks (e.g. fulcrum pins may be made to engage hooks which hold them into position) or via bonding or the like.

In still further embodiments, the dual elongated fulcrums supporting the carriage may be replaced with a single central pivot element that allows the carriage to undergo a two dimensional motion during actuation instead of a one dimensional motion. In still other embodiments an additional control element (mechanically, electrically, or magnetically actuated) may be used to provide a variation in the scanning amplitude either dynamically or statically.

### FURTHER ALTERNATIVES AND CONCLUSIONS

In some embodiments, the formation of the devices or structures may include various post layer formation operations. Some such post layer formation operations may include transferring the device from a temporary substrate to another substrate. Some embodiments may employ diffusion bonding or the like to enhance adhesion between successive layers of material. Various teachings concerning the use of diffusion bonding in electrochemical fabrication process is set forth in U.S. Patent Application No. 60/534,204 which was filed Dec. 31, 2003 by Cohen et al. which is entitled “Method for Fabricating Three-Dimensional Structures Including Surface Treatment of a First Material in Preparation for Deposition of a Second Material”; U.S. patent application Ser. No. 10/841,382, filed May 7, 2004 by Zhang, et al., and which is entitled “Method of Electrochemically Fabricating Multilayer Structures Having Improved Interlayer Adhesion”; U.S. patent application Ser. No. 10/841,384, filed May 7, 2004 by Zhang, et al., and which is entitled “Method of Electrochemically Fabricating Multilayer Structures Having Improved Interlayer Adhesion”. Each of these applications is incorporated herein by reference as if set forth in full.

As noted above, the formation of devices or structures as set forth herein may involve a use of structural or sacrificial dielectric materials. Additional teachings concerning the formation of structures on dielectric substrates and/or the formation of structures that incorporate dielectric materials into the formation process and possibly into the final structures as formed are set forth in a number of patent applications filed Dec. 31, 2003. The first of these filings is U.S. Patent Application No. 60/534,184 which is entitled “Electrochemical Fabrication Methods Incorporating Dielectric Materials and/or Using Dielectric Substrates”. The second of these filings is U.S. Patent Application No. 60/533,932, which is entitled “Electrochemical Fabrication Methods Using Dielectric Substrates”. The third of these filings is U.S. Patent Application No. 60/534,157, which is entitled “Electrochemical Fabrication Methods Incorporating Dielectric Materials”. The fourth of these filings is U.S. Patent Application No. 60/533,891, which is entitled “Methods for Electrochemically Fabricating Structures Incorporating Dielectric Sheets and/or Seed layers That Are Partially Removed Via Planarization”. A fifth such filing is U.S. Patent Application No. 60/533,895, which is entitled “Electrochemical Fabrication Method for Producing Multi-layer Three-Dimensional Structures on a Porous Dielectric”. Additional patent filings that provide teachings concerning incorporation of dielectrics into the EFAB process include U.S. patent application Ser. No. 11/139,262, filed May 26, 2005 by Lockard, et al., and which is entitled “Methods for Electrochemically Fabricating Structures Using Adhered Masks, Incorporating Dielectric Sheets, and/or Seed Layers that are Partially Removed Via Planarization”; and U.S. patent application Ser. No. 11/029,216, filed Jan. 3, 2005 by Cohen, et al., and which is entitled “Electrochemical Fabrication Methods Incorporating Dielectric Materials and/or Using Dielectric Substrates”. These patent filings are each hereby incorporated herein by reference as if set forth in full herein.

Further teachings about planarizing layers and setting layers thicknesses and the like are set forth in the following US Patent Applications which were filed Dec. 31, 2003: (1) U.S. Patent Application No. 60/534,159 by Cohen et al. and which is entitled “Electrochemical Fabrication Methods for Producing Multilayer Structures Including the use of Diamond Machining in the Planarization of Deposits of Material” and (2) U.S. Patent Application No. 60/534,183 by Cohen et al. and which is entitled “Method and Apparatus for Maintaining Parallelism of Layers and/or Achieving Desired Thicknesses of Layers During the Electrochemical Fabrication of Structures”. An additional filing that provides teachings related to planarization are found in U.S. patent application Ser. No. 11/029,220, filed Jan. 3, 2005 by Frodis, et al., and which is entitled “Method and Apparatus for Maintaining Parallelism of Layers and/or Achieving Desired Thicknesses of Layers During the Electrochemical Fabrication of Structures”. These patent filings are each hereby incorporated herein by reference as if set forth in full herein.

Though the embodiments explicitly set forth herein have considered multi-material layers to be formed one after another. As noted herein some post layer formation assembly can occur. Post layer assembly may involve assembly of split structures as taught in U.S. patent application Ser. No. 11/506,586; packaging and alignment methods taught in U.S. patent application Ser. No. 11/685,118; and/or adding on of additional materials as taught in U.S. patent application Ser. No. 10/841,001. Each of these applications is incorporated herein by reference as if set forth in full.

Still other alternative embodiments may make use of fabrication techniques taught in U.S. patent application Ser. No. 10/949,744 for forming gaps and structural features which are smaller than a minimum feature size dictated by the fabrication process under reasonable formation conditions; and Ser. No. 11/441,578 for forming bearings and bushings. Each of these applications is incorporated herein by reference as if set forth in full.

In preferred embodiments of the invention, the devices are preferably made from metal (e.g., nickel-cobalt, nickel-titanium, nickel phosphorous, nickel titanium, stainless steel) and are preferably produced using a multi-layer micro-manufacturing process such an electrochemical fabrication process described herein above. Additional information about electrochemically forming structures that contain nickel titanium and other non-platable materials may be found in U.S. patent application Ser. No. 11/478,934, filed Jun. 26, 2006, which is hereby incorporated herein by reference as if set forth in full. In other embodiments, other materials may be used or incorporated into the devices and other fabrication processes may be used.

Though various portions of this specification have been provided with headers, it is not intended that the headers be used to limit the application of teachings found in one portion of the specification from applying to other portions of the specification. For example, it should be understood that alternatives acknowledged in association with one embodiment, are intended to apply to all embodiments to the extent that the features of the different embodiments make such application functional and do not otherwise contradict or remove all benefits of the adopted embodiment. Various other embodiments of the present invention exist. Some of these embodiments may be based on a combination of the teachings herein with various teachings incorporated herein by reference.

Many other alternative embodiments will be apparent to those of skill in the art upon review or the teachings herein. Further embodiments may be formed from a combination of the various teachings explicitly set forth in the body of this application. Even further embodiments may be formed by combining the teachings set forth explicitly herein with teachings set forth in the various applications and patents referenced herein, each of which is incorporated herein by reference. In view of the teachings herein, many further embodiments, alternatives in design and uses of the instant invention will be apparent to those of skill in the art. As such, it is not intended that the invention be limited to the particular illustrative embodiments, alternatives, and uses described above but instead that it be solely limited by the claims presented hereafter. While the multi-layer electrochemical fabrication processes are preferred, other manufacturing processes may be used to form the devices, or portions of the devices set forth herein.

I claim:

1. An intravascular imaging device, comprising:
a catheter having a proximal and distal end;
a scanning mechanism having an proximal end and a distal end, wherein the proximal is functionally connected to the distal end of the catheter;
a drive cable located within the catheter and functionally connected to scanning mechanism;
wherein the scanning mechanism comprises:
a signal tranducer,
a mechanical mechanism for scanning the transducer that caused the scanning mechanism to scan a pattern which is different from a motion carried by the drive cable.
